(19) United States
(12) Patent Application Publication
(10) Pub. No.: US 2015/0232870 A1
(43) Pub. Date: Aug. 20, 2015

(54) TRANSGENIC PLANTS WITH ENHANCED AGRONOMIC TRAITS

(71) Applicant: MONSANTO TECHNOLOGY LLC, ST. LOUIS, MO (US)

(72) Inventors: MARK SCOTT ABAD, WEBSTER GROVES, MO (US); MURTAZA F. ALIBHAI, ST. LOUIS, MO (US); ALICE CLARA AUGUSTINE, BANGALORE (IN); AMARJIT BASRA, ST. LOUIS, MO (US); ERIN BELL, ST. LOUIS, MO (US); TERRY BRADSHAW, NEWTON, KS (US); PAOLO CASTIGLIONI, DAVIS, CA (US); JAISHREE M. CHITTOOR-VIJAYANATH, WILDWOOD, MO (US); JILL DEIKMAN, DAVIS, CA (US); MOLIAN DENG, GROVER, MO (US); MICHAEL D. EDGERTON, ST. LOUIS, MO (US); KAREN GABBERT, ST. LOUIS, MO (US); BARRY S. GOLDMAN, ST. LOUIS, MO (US); BALASULOJINI KARUNANANDAA, CREVE COEUR, MO (US); SUSANNE KJEMTRUP-LOVELACE, ST. LOUIS, MO (US); TIMOTHY J. LELAND, ST. LOUIS, MI (US); ADRIAN LUND, HALSTEAD, KS (US); LINDA L. LUTFIYYA, ST. LOUIS, MA (US); MARCUS MCNABNAY, EDWARDSVILLE, IL (US); DONALD E. NELSON, DURHAM, NC (US); THOMAS G. RUFF, WILDWOOD, MO (US); BETH SAVIDGE, DAVIS, CA (US); PAUL SCHAFFER, STONINGTON, CT (US); PADMINI SUDARSHANA, BANGALORE (IN); K. SUMATHY, ST. LOUIS, MO (US); CAROLYN THAI, O'FALLON, MO (US); REBECCA L. THOMPSON-MIZE, ST. CHARLES, MO (US); CARL P. URWIN, DARDENNE PRAIRIE, MO (US); H. G. VEENA, TOERNOOIVLED 1, 6525 ED NIJMEGEN (NL); TYMAGONDLU V. VENKATESH, ST. LOUIS, MO (US); STEVEN T. VOSS, EDWARDSVILLE, IL (US); ZHIDONG XIE, MARYLAND HEIGHTS, MO (US)

(21) Appl. No.: 14/544,461

(22) Filed: Jan. 9, 2015

Related U.S. Application Data
(63) Continuation of application No. 12/218,975, filed on Jul. 18, 2008, now abandoned.
(60) Provisional application No. 60/961,192, filed on Jul. 19, 2007.

(51) Int. Cl. C12N 15/82; A01H 1/04

(52) U.S. Cl. CPC C12N 15/8247; C12N 15/8273; C12N 15/8261; C12N 15/8251; A01H 1/04

(57) ABSTRACT

This invention provides transgenic plant cells with recombinant DNA for expression of proteins that are useful for imparting enhanced agronomic trait(s) to transgenic crop plants. This invention also provides transgenic plants and progeny seed comprising the transgenic plant cells where the plants are selected for having an enhanced trait selected from the group of traits consisting of enhanced water use efficiency, enhanced cold tolerance, increased yield, enhanced nitrogen use efficiency, enhanced seed protein and enhanced seed oil. Also disclosed are methods for manufacturing transgenic seed and plants with enhanced trait.

### CROSS REFERENCE TO RELATED APPLICATIONS

This application is a continuation application of U.S. Ser. No. 12/218,975, filed Jul. 18, 2008, which is a non-provisional application and claims benefit of priority to and of U.S. provisional application Ser. No. 60/961,192, filed Jul. 19, 2007 herein incorporated by reference in its entirety.

### INCORPORATION OF SEQUENCE LISTING

Two copies of the sequence listing (Copy #1 and Copy #2) and a computer readable form (CRF) of the sequence listing, all on CD-Rs, each containing the text file named 3126016US2.txt, which is 6,332,416 bytes (measured in MS-WINDOWS), were created on Jan. 9, 2015 and are herein incorporated by reference.

### INCORPORATION OF COMPUTER PROGRAM LISTING

Two copies of the Computer Program Listing (Copy 1 and Copy 2) and a computer readable form (CRF) containing folders hmmer-2.3.2 and 32pfamDir, all on CD-Rs are incorporated herein by reference in their entirety. Folder hmmer-2.3.2 contains the source code and other associated files for implementing the HMMer software for Pfam analysis. Folder 32pfamDir contains 32 Pfam Hidden Markov Models. Both folders were created on CD-R on Jan. 9, 2015, having a total size of 2,031,616 (measured in MS-WINDOWS).

### FIELD OF THE INVENTION

Disclosed herein are recombinant DNA useful for providing enhanced traits to transgenic plants, seeds, pollen, plant cells and plant nuclei of such transgenic plants, methods of making and using such recombinant DNA, plants, seeds, pollen, plant cells and plant nuclei. Also disclosed are methods of producing hybrid seed comprising such recombinant DNA.

### BACKGROUND OF THE INVENTION

This invention employs recombinant DNA for expression of proteins that are useful for imparting enhanced agronomic traits to transgenic plants. Recombinant DNA in this invention is provided in a construct comprising a promoter that is functional in plant cells and that is operably linked to a DNA segment that encodes a protein. In some embodiments of the invention, such protein defined by protein domains e.g. a “Pfam domain module” (as defined herein below) from the group of Pfam domain modules identified in Table 10. In other embodiments of the invention, e.g. where a Pfam domain module is not available, such protein is defined a consensus amino acid sequence of an encoded protein that is targeted for production e.g. a protein having amino acid sequence with at least 90% identity to a consensus amino acid sequence as set forth in SEQ ID NO: 2201. In more specific embodiments of the invention the protein expressed in plant cells is a protein selected from the group of proteins identified in Table 1 and their homologs identified in Table 8.

Other aspects of the invention are specifically directed to plant cell nuclei and transgenic plant cells comprising the recombinant DNA construct of the invention, transgenic plants comprising a plurality of such plant cells, progeny transgenic seed, embryo and transgenic pollen from such transgenic plants. Such transgenic plants are selected from a population of transgenic plants regenerated from plant cells transformed with the recombinant DNA construct provided by the invention and express the protein by screening transgenic plants in the population for an enhanced trait as compared to control plants that do not have the recombinant DNA construct, where the enhanced trait is selected from group of enhanced traits consisting of enhanced water use efficiency, enhanced cold tolerance, increased yield, enhanced nitrogen use efficiency, enhanced seed protein and enhanced seed oil.

In yet another aspect of the invention the plant cell nuclei, plant cells, transgenic plants, seeds, and pollen further comprise recombinant DNA expressing a protein that provides tolerance from exposure to an herbicide applied at levels that are lethal to a wild type plant cell. Such tolerance is especially useful not only as an advantageous trait in such plants but is also useful in a selection step in the methods of the invention. In aspects of the invention such herbicide is a glyphosate, dicamba, or glufosinate compound.

Yet other aspects of the invention provide transgenic plants which are homozygous for the recombinant DNA and transgenic seed of the invention from corn, soybean, cotton, canola, alfalfa, wheat or rice plants.

This invention also provides methods for manufacturing non-natural, transgenic seed that can be used to produce a crop of transgenic plants with an enhanced Wait resulting from expression of stably-integrated, recombinant DNA construct provided by herein. More specifically the method comprises (a) providing a population of plants produced from a parental plant having a recombinant DNA construct of the invention; (b) screening this population of plants for at least one enhanced trait and the recombinant DNA construct, where individual plants in the population can exhibit the trait at a level less than, essentially the same as or greater than the level that the trait is exhibited in control plants which do not contain the recombinant DNA construct, where the enhanced trait is selected from the group of enhanced traits consisting of enhanced water use efficiency, enhanced cold tolerance, enhanced heat tolerance, enhanced high salinity tolerance, enhanced shade tolerance, increased yield, enhanced nitrogen use efficiency, enhanced seed protein and enhanced seed oil; (c) selecting from the population one or more plants that exhibit the trait at a level greater than the level that the trait is exhibited in control plants; and (d) collecting seeds from selected plant selected from step c. The method further comprises (e) verifying that the recombinant DNA construct is stably integrated in said selected plants, and (f) analyzing tissue of a selected plant to determine the production of a protein having the function of a protein selected from SEQ ID NO: 96 through SEQ ID NO: 2166. In one aspect of the invention the plants in the population further comprise DNA expressing a protein that provides tolerance to exposure to a herbicide applied at levels that are lethal to wild type plant cells and the selecting is affected by treating the population with the herbicide, e.g. a glyphosate, dicamba, or glufosinate compound. In another aspect of the invention the plants are selected by identifying plants with the enhanced trait. The methods are especially useful for manufacturing corn, soybean, cotton, canola, alfalfa, wheat or rice seed.

Another aspect of the invention provides a method of producing hybrid corn seed comprising acquiring hybrid corn seed from a herbicide tolerant corn plant which also has stably-integrated, recombinant DNA construct comprising a promoter that is (a) functional in plant cells and (b) is operably linked to DNA that encodes a protein provided by the invention. The methods further comprise producing corn plants from the hybrid corn seed, wherein a fraction of the plants produced from the hybrid corn seed is homozygous for the recombinant DNA, a fraction of the plants produced from the hybrid corn seed is hemizygous for the recombinant DNA construct, and a fraction of the plants produced from the hybrid corn seed has none of the recombinant DNA construct; selecting corn plants which are homozygous and hemizygous for the recombinant DNA construct by treating with an herbicide; collecting seed from herbicide-treated-surviving corn plants and planting the seed to produce further progeny corn plants; repeating the selecting and collecting steps at least once to produce an inbred corn line; and crossing the inbred corn line with a second corn line to produce hybrid seed.

### BRIEF DESCRIPTION OF THE DRAWINGS

FIG. 1 is a consensus amino acid sequence of SEQ ID NO: 127 and its homologs.

FIGS. 2-5 are plasmid maps.

### DETAILED DESCRIPTION OF THE INVENTION

In the attached sequence listing:

SEQ ID NO:1-95 are nucleotide sequences of the coding strand of DNA for “genes” used in the recombinant DNA imparting an enhanced trait in plant cells, i.e. each represents a coding sequence for a protein;

SEQ ID NO: 96-193 are amino acid sequences of the cognate protein of the “genes” with nucleotide coding sequences 1-95;

SEQ ID NO: 194-2166 are amino acid sequences of homologous proteins;

SEQ ID NO: 2167-2200 are nucleotide sequences of the elements in base plasmid vectors

SEQ ID NO: 2201 is a consensus amino acid sequence.

SEQ ID NO: 2202-2203 are nucleotide sequences of two base plasmid vectors useful for corn transformation;

SEQ ID NO: 2204 is a nucleotide sequence of a base plasmid vector useful for soybean and canola transformation; and

SEQ ID NO: 2205 is a nucleotide sequence of a base plasmid vector useful for cotton transformation.

As used herein a “plant cell” means a plant cell that is transformed with stably-integrated, non-natural, recombinant DNA, e.g. by Agrobacterium-mediated transformation or by bombardment using microparticles coated with recombinant DNA or other means. A plant cell of this invention can be an originally-transformed plant cell that exists as a microorganism or as a progeny plant cell that is regenerated into differentiated tissue, e.g. into a transgenic plant with stably-integrated, non-natural recombinant DNA, or seed or pollen derived from a progeny transgenic plant.

As used herein a “transgenic plant” means a plant whose genome has been altered by the stable integration of recombinant DNA. A transgenic plant includes a plant regenerated from an originally-transformed plant cell and progeny transgenic plants from later generations or crosses of a transformed plant.

As used herein “recombinant DNA” means DNA which has been genetically engineered and constructed outside of a cell including DNA containing naturally occurring DNA or cDNA or synthetic DNA.

As used herein “consensus sequence” means an artificial sequence of amino acids in a conserved region of an alignment of amino acid sequences of homologous proteins, e.g. as determined by a CLUSTALW alignment of amino acid sequence of homolog proteins.

As used herein “homolog” means a protein in a group of proteins that perform the same biological function, e.g. proteins that belong to the same Pfam protein family and that provide a common enhanced trait in transgenic plants of this invention. Homologs are expressed by homologous genes. Homologous genes include naturally occurring alleles and artificially-created variants. Degeneracy of the genetic code provides the possibility to substitute at least one base of the protein encoding sequence of a gene with a different base without causing the amino acid sequence of the polypeptide produced from the gene to be changed. Hence, a polynucleotide useful in the present invention may have any base sequence that has been changed from SEQ ID NO: 1 through SEQ ID NO: 95 in accordance with degeneracy of the genetic code. Homologs are proteins that, when optimally aligned, have at least 60% identity, more preferably about 70% or higher, more preferably at least 80% and even more preferably at least 90% identity over the full length of a protein identified as being associated with imparting an enhanced trait when expressed in plant cells. Homologs include proteins with an amino acid sequence that has at least 90% identity to a consensus amino acid sequence of proteins and homologs disclosed herein.

Homologs are identified by comparison of amino acid sequence, e.g. manually or by use of a computer-based tool using known homology-based search algorithms such as those commonly known and referred to as BLAST, FASTA, and Smith-Waterman. A local sequence alignment program, e.g. BLAST, can be used to search a database of sequences to find similar sequences, and the summary Expectation value (E-value) used to measure the sequence base similarity. As a protein hit with the best E-value for a particular organism may not necessarily be an ortholog or the only ortholog, a reciprocal query is used in the present invention to filter hit sequences with significant E-values for ortholog identification. The reciprocal query entails search of the significant hits against a database of amino acid sequences from the base organism that are similar to the sequence of the query protein. A hit is a likely ortholog, when the reciprocal query's best hit is the query protein itself or a protein encoded by a duplicated gene after speciation. A further aspect of the invention comprises functional homolog proteins that differ in one or more amino acids from those of disclosed protein as the result of conservative amino acid substitutions, for example substitutions are among: acidic (negatively charged) amino acids such as aspartic acid and glutamic acid; basic (positively charged) amino acids such as arginine, histidine, and lysine; neutral polar amino acids such as glycine, serine, threonine, cysteine, tyrosine, asparagine, and glutamine; neutral nonpolar (hydrophobic) amino acids such as alanine, leucine, isoleucine, valine, proline, phenylalanine, tryptophan, and methionine; amino acids having aliphatic side chains such as glycine, alanine, valine, leucine, and isoleucine; amino acids having aliphatic-hydroxyl side chains such as serine and threonine; amino acids having amide-containing side chains such as asparagine and glutamine; amino acids having aromatic side chains such as phenylalanine, tyrosine, and tryptophan; amino acids having basic side chains such as lysine, arginine, and histidine; amino acids having sulfur-containing side chains such as cysteine and methionine; naturally conservative amino acids such as valine-leucine, valine-isoleucine, phenylalanine-tyrosine, lysine-arginine, alanine-valine, aspartic acid-glutamic acid, and asparagine-glutamine. A further aspect of the homologs encoded by DNA useful in the transgenic plants of the invention are those proteins that differ from a disclosed protein as the result of deletion or insertion of one or more amino acids in a native sequence.

“Percent identity” describes the extent to which the sequences of DNA or protein segments are invariant throughout a window of alignment of nucleotide or amino acid sequences. An “identity fraction” for a sequence aligned with a reference sequence is the number of identical components which are shared by the sequences, divided by a length of the window of alignment, wherein the length does not include gaps introduced by an alignment algorithm. “Percent identity” (“% identity”) is the identity fraction times 100. The alignment algorithm is preferably a local alignment algorithm, such as BLASTp. As used herein, sequences are “aligned” when the alignment produced by BLASTp has a minimal e-value.

“Pfam” database is a large collection of multiple sequence alignments and hidden Markov models covering many common protein families, e.g. Pfam version 19.0 (December 2005) contains alignments and models for 8183 protein families and is based on the Swissprot 47.0 and SP-TrEMBL 30.0 protein sequence databases. See S. R. Eddy, “Profile Hidden Markov Models”, Bioinformatics 14:755-763, 1998. The Pfam database is currently maintained and updated by the Pfam Consortium. The alignments represent some evolutionary conserved structure that has implications for the protein's function. Profile hidden Markov models (profile HMMs) built from the protein family alignments are useful for automatically recognizing that a new protein belongs to an existing protein family even if the homology by alignment appears to be low.

Protein domains are identified by querying the amino acid sequence of a protein against Hidden Markov Models which characterize protein family domains (“Pfam domains”) using HMMER software, a current version of which is provided in the appended computer listing. A protein domain meeting the gathering cutoff for the alignment of a particular Pram domain is considered to contain the Pram domain.

A “Pram domain module” is a representation of Pfam domains in a protein, in order from N terminus to C terminus. In a Pfam domain module individual Pfam domains are separated by double colons “::”. The order and copy number of the Pfam domains from N to C terminus are attributes of a Pfam domain module. Although the copy number of repetitive domains is important, varying copy number often enables a similar function. Thus, a Pfam domain module with multiple copies of a domain should define an equivalent Pram domain module with variance in the number of multiple copies. A Pfam domain module is not specific for distance between adjacent domains, but contemplates natural distances and variations in distance that provide equivalent function. The Pfam database contains both narrowly- and broadly-defined domains, leading to identification of overlapping domains on some proteins. A Pfam domain module is characterized by non-overlapping domains. Where there is overlap, the domain having a function that is more closely associated with the function of the protein (based on the E value of the Pfam match) is selected. One protein is identified as containing a pfam domain when its scores is higher than the gathering cutoff disclosed in Table 12 by Pfam analysis disclosed herein

Once one DNA is identified as encoding a protein which imparts an enhanced trait when expressed in transgenic plants, other DNA encoding proteins with the same Pfam domain module are identified by querying the amino acid sequence of protein encoded by candidate DNA against the Hidden Markov Models which characterizes the Pfam domains using HMMER software. Candidate proteins meeting the same Pfam domain module are in the protein family and have cognate DNA that is useful in constructing recombinant DNA for the use in the plant cells of this invention. Hidden Markov Model databases for use with HMMER software in identifying DNA expressing protein with a common Pfam domain module for recombinant DNA in the plant cells of this invention are also included in the appended computer listing.

The HMMER software and Pfam databases are version 19.0 and were used to identify known domains in the proteins corresponding to amino acid sequence of SEQ ID NO: 96 through SEQ ID NO: 193. All DNA encoding proteins that have at least one of pfam domain modules of this invention can be used in recombinant DNA construct of this invention, e.g. for selecting transgenic plants having enhanced agronomic traits. The relevant Pfams modules for use in this invention, as more specifically disclosed below, are Homeobox, Myb_DNA-binding::Myb_{—}DNA-binding, Myb_DNA-binding, zf-Dof, zf-C2H2::zf-C2H2, AP2, Response_reg::Myb_DNA-binding, B3, B3::Auxin_resp::AUX_IAA, HLH, NAM, B3::B3, AUX_IAA, KNOX1::KNOX2::ELK, GRAS, AT_hook::AT_HOOK::DUF296, TCP, SBP, zf-C2H2, B3::Auxin_resp, EIN3, bZIP_{—}2, zf-B_box::zf-B_box, zf-B_box::CCT, RWP-RK::PB1, F-box::TUB, CBFD_NFYB_HMF, GATA, SRF-TF, K-box, and SRF-TF::K-box.

As used herein “promoter” means regulatory DNA for initializing transcription. A “plant promoter” is a promoter capable of initiating transcription in plant cells whether or not its origin is a plant cell, e.g. it is well known that Agrobacterium promoters are functional in plant cells. Thus, plant promoters include promoter DNA obtained from plants, plant viruses and bacteria such as Agrobacterium and Bradyrhizobium bacteria. Examples of promoters under developmental control include promoters that preferentially initiate transcription in certain tissues, such as leaves, roots, or seeds. Such promoters are referred to as “tissue preferred”. Promoters that initiate transcription only in certain tissues are referred to as “tissue specific”. A “cell type” specific promoter primarily drives expression in certain cell types in one or more organs, for example, vascular cells in roots or leaves. An “inducible” or “repressible” promoter is a promoter which is under environmental control. Examples of environmental conditions that may affect transcription by inducible promoters include anaerobic conditions, or certain chemicals, or the presence of light. Tissue specific, tissue preferred, cell type specific, and inducible promoters constitute the class of “non-constitutive” promoters. A “constitutive” promoter is a promoter which is active under most conditions.

As used herein “operably linked” means the association of two or more DNA fragments in a DNA construct so that the function of one, e.g. protein-encoding DNA, is controlled by the other, e.g. a promoter.

As used herein “expressed” means produced, e.g. a protein is expressed in a plant cell when its cognate DNA is transcribed to mRNA that is translated to the protein.

As used herein a “control plant” means a plant that does not contain the recombinant DNA that expresses a protein that imparts an enhanced trait. A control plant is to identify and select a transgenic plant that has an enhance trait. A suitable control plant can be a non-transgenic plant of the parental line used to generate a transgenic plant, i.e. devoid of recombinant DNA. A suitable control plant may in some cases be a progeny of a hemizygous transgenic plant line that is does not contain the recombinant DNA, known as a negative segregant.

As used herein an “enhanced trait” means a characteristic of a transgenic plant that includes, but is not limited to, an enhance agronomic trait characterized by enhanced plant morphology, physiology, growth and development, yield, nutritional enhancement, disease or pest resistance, or environmental or chemical tolerance. In more specific aspects of this invention enhanced trait is selected from group of enhanced traits consisting of enhanced water use efficiency, enhanced cold tolerance, increased yield, enhanced nitrogen use efficiency, enhanced seed protein and enhanced seed oil. In an important aspect of the invention the enhanced trait is enhanced yield including increased yield under non-stress conditions and increased yield under environmental stress conditions. Stress conditions may include, for example, drought, shade, fungal disease, viral disease, bacterial disease, insect infestation, nematode infestation, cold temperature exposure, heat exposure, osmotic stress, reduced nitrogen nutrient availability, reduced phosphorus nutrient availability and high plant density. “Yield” can be affected by many properties including without limitation, plant height, pod number, pod position on the plant, number of internodes, incidence of pod shatter, grain size, efficiency of nodulation and nitrogen fixation, efficiency of nutrient assimilation, resistance to biotic and abiotic stress, carbon assimilation, plant architecture, resistance to lodging, percent seed germination, seedling vigor, and juvenile traits. Yield can also be affected by efficiency of germination (including germination in stressed conditions), growth rate (including growth rate in stressed conditions), ear number, seed number per ear, seed size, composition of seed (starch, oil, protein) and characteristics of seed fill.

Increased yield of a transgenic plant of the present invention can be measured in a number of ways, including test weight, seed number per plant, seed weight, seed number per unit area (i.e. seeds, or weight of seeds, per acre), bushels per acre, tones per acre, tons per acre, kilo per hectare. For example, maize yield may be measured as production of shelled corn kernels per unit of production area, for example in bushels per acre or metric tons per hectare, often reported on a moisture adjusted basis, for example at 15.5 percent moisture. Increased yield may result from improved utilization of key biochemical compounds, such as nitrogen, phosphorous and carbohydrate, or from improved responses to environmental stresses, such as cold, heat, drought, salt, and attack by pests or pathogens. Recombinant DNA used in this invention can also be used to provide plants having improved growth and development, and ultimately increased yield, as the result of modified expression of plant growth regulators or modification of cell cycle or photosynthesis pathways. Also of interest is the generation of transgenic plants that demonstrate enhanced yield with respect to a seed component that may or may not correspond to an increase in overall plant yield. Such properties include enhancements in seed oil, seed molecules such as tocopherol, protein and starch, or oil particular oil components as may be manifest by alterations in the ratios of seed components.

A subset of the DNA molecules of this invention includes fragments of the disclosed recombinant DNA consisting of oligonucleotides of at least 15, preferably at least 16 or 17, more preferably at least 18 or 19, and even more preferably at least 20 or more, consecutive nucleotides. Such oligonucleotides are fragments of the larger molecules having a sequence selected from the group consisting of SEQ ID NO: 1 through SEQ ID NO: 95, and find use, for example as probes and primers for detection of the polynucleotides of the present invention.

Recombinant DNA constructs are assembled using methods well known to persons of ordinary skill in the art and typically comprise a promoter operably linked to DNA, the expression of which provides the enhanced agronomic trait. Other construct components may include additional regulatory elements, such as 5′ leaders and introns for enhancing transcription, 3′ untranslated regions (such as polyadenylation signals and sites), DNA for transit or signal peptides.

Numerous promoters that are active in plant cells have been described in the literature. These include promoters present in plant genomes as well as promoters from other sources, including nopaline synthase (NOS) promoter and octopine synthase (OCS) promoters carried on tumor-inducing plasmids of Agrobacterium tumefaciens and the CaMV35S promoters from the cauliflower mosaic virus as disclosed in U.S. Pat. Nos. 5,164,316 and 5,322,938. Useful promoters derived from plant genes are found in U.S. Pat. No. 5,641,876, which discloses a rice actin promoter, U.S. Pat. No. 7,151,204, which discloses a maize chloroplast aldolase promoter and a maize aldolase (FDA) promoter, and U.S. Patent Application Publication 2003/0131377 A1, which discloses a maize nicotianamine synthase promoter, all of which are incorporated herein by reference. These and numerous other promoters that function in plant cells are known to those skilled in the art and available for use in recombinant polynucleotides of the present invention to provide for expression of desired genes in transgenic plant cells.

In other aspects of the invention, preferential expression in plant green tissues is desired. Promoters of interest for such uses include those from genes such as Arabidopsis thaliana ribulose-1,5-bisphosphate carboxylase (Rubisco) small subunit (Fischhoff et al. (1992) Plant Mol Biol. 20:81-93), aldolase and pyruvate orthophosphate dikinase (PPDK) (Taniguchi et al. (2000) Plant Cell Physiol. 41(1):42-48).

Furthermore, the promoters may be altered to contain multiple “enhancer sequences” to assist in elevating gene expression. Such enhancers are known in the art. By including an enhancer sequence with such constructs, the expression of the selected protein may be enhanced. These enhancers often are found 5′ to the start of transcription in a promoter that functions in eukaryotic cells, but can often be inserted upstream (5′) or downstream (3′) to the coding sequence. In some instances, these 5′ enhancing elements are introns. Particularly useful as enhancers are the 5′ introns of the rice actin 1 (see U.S. Pat. No. 5,641,876) and rice actin 2 genes, the maize alcohol dehydrogenase gene intron, the maize heat shock protein 70 gene intron (U.S. Pat. No. 5,593,874) and the maize shrunken 1 gene.

In other aspects of the invention, sufficient expression in plant seed tissues is desired to affect improvements in seed composition. Exemplary promoters for use for seed composition modification include promoters from seed genes such as napin (U.S. Pat. No. 5,420,034), maize L3 oleosin (U.S. Pat. No. 6,433,252), zein Z27 (Russell et al. (1997) Transgenic Res. 6(2):157-166); globulin 1 (Belanger et al (1991) Genetics 129:863-872), glutelin 1 (Russell (1997) supra), and peroxiredoxin antioxidant (Per1) (Stacy et al. (1996) Plant Mol Biol. 31(6):1205-1216).

Recombinant DNA constructs prepared in accordance with the invention will also generally include a 3′ element that typically contains a polyadenylation signal and site. Well-known 3′ elements include those from Agrobacterium tumefaciens genes such as nos 3′, tml 3′, tmr 3′, tms 3′, ocs 3′, tr7 3′, for example disclosed in U.S. Pat. No. 6,090,627, incorporated herein by reference; 3′ elements from plant genes such as wheat (Triticum aesevitum) heat shock protein 17 (Hsp17 3′), a wheat ubiquitin gene, a wheat fructose-1,6-biphosphatase gene, a rice glutelin gene, a rice lactate dehydrogenase gene and a rice beta-tubulin gene, all of which are disclosed in U.S. published patent application 2002/0192813 A1, incorporated herein by reference; and the pea (Pisum sativum) ribulose biphosphate carboxylase gene (rbs 3′), and 3′ elements from the genes within the host plant.

Constructs and vectors may also include a transit peptide for targeting of a gene to a plant organelle, particularly to a chloroplast, leucoplast or other plastid organelle. For descriptions of the use of chloroplast transit peptides see U.S. Pat. No. 5,188,642 and U.S. Pat. No. 5,728,925, incorporated herein by reference. For description of the transit peptide region of an Arabidopsis EPSPS gene useful in the present invention, see Klee, H. J. et al (MGG (1987) 210:437-442).

Transgenic plants comprising or derived from plant cells of this invention transformed with recombinant DNA construct can be further enhanced with stacked traits, e.g. a crop plant having an enhanced trait resulting from expression of DNA disclosed herein in combination with herbicide and/or pest resistance traits. For example, genes of the current invention can be stacked with other traits of agronomic interest, such as a trait providing herbicide resistance, or insect resistance, such as using a gene from Bacillus thuringensis to provide resistance against lepidopteran, coliopteran, homopteran, hemiopteran, and other insects. Herbicides for which transgenic plant tolerance has been demonstrated and the method of the present invention can be applied include, but are not limited to, glyphosate, dicamba, glufosinate, sulfonylurea, bromoxynil and norflurazon herbicides. Polynucleotide molecules encoding proteins involved in herbicide tolerance are well-known in the art and include, but are not limited to, a polynucleotide molecule encoding 5-enolpyruvylshikimate-3-phosphate synthase (EPSPS) disclosed in U.S. Pat. Nos. 5,094,945; 5,627,061; 5,633,435 and 6,040,497 for imparting glyphosate tolerance; polynucleotide molecules encoding a glyphosate oxidoreductase (GOX) disclosed in U.S. Pat. No. 5,463,175 and a glyphosate-N-acetyl transferase (GAT) disclosed in U.S. Patent Application publication 2003/0083480 A1 also for imparting glyphosate tolerance; dicamba monooxygenase disclosed in U.S. Patent Application publication 2003/0135879 A1 for imparting dicamba tolerance; a polynucleotide molecule encoding bromoxynil nitrilase (Bxn) disclosed in U.S. Pat. No. 4,810,648 for imparting bromoxynil tolerance; a polynucleotide molecule encoding phytoene desaturase (crtl) described in Misawa et al, (1993) Plant J. 4:833-840 and in Misawa et al, (1994) Plant J. 6:481-489 for norflurazon tolerance; a polynucleotide molecule encoding acetohydroxyacid synthase (AHAS, aka ALS) described in Sathasiivan et al. (1990) Nucl. Acids Res. 18:2188-2193 for imparting tolerance to sulfonylurea herbicides; polynucleotide molecules known as bar genes disclosed in DeBlock, et al. (1987) EMBO J. 6:2513-2519 for imparting glufosinate and bialaphos tolerance; polynucleotide molecules disclosed in U.S. Patent Application Publication 2003/010609 A1 for imparting N-amino methyl phosphonic acid tolerance; polynucleotide molecules disclosed in U.S. Pat. No. 6,107,549 for impartinig pyridine herbicide resistance; molecules and methods for imparting tolerance to multiple herbicides such as glyphosate, atrazine, ALS inhibitors, isoxoflutole and glufosinate herbicides are disclosed in U.S. Pat. No. 6,376,754 and U.S. Patent Application Publication 2002/0112260, all of said U.S. patents and patent application Publications are incorporated herein by reference. Molecules and methods for imparting insect/nematode/virus resistance are disclosed in U.S. Pat. Nos. 5,250,515; 5,880,275; 6,506,599; 5,986,175 and U.S. Patent Application Publication 2003/0150017 A1, all of which are incorporated herein by reference.

### Plant Cell Transformation Methods

Numerous methods for transforming plant cells with recombinant DNA construct are known in the art and may be used in the present invention. Two commonly used methods for plant transformation are Agrobacterium-mediated transformation and microprojectile bombardment. Microprojectile bombardment methods are illustrated in U.S. Pat. No. 5,015,580 (soybean); U.S. Pat. No. 5,550,318 (corn); U.S. Pat. No. 5,538,880 (corn); U.S. Pat. No. 5,914,451 (soybean); U.S. Pat. No. 6,160,208 (corn); U.S. Pat. No. 6,399,861 (corn), U.S. Pat. No. 6,153,812 (wheat) and U.S. Pat. No. 6,365,807 (rice), and Agrobacterium-mediated transformation is described in U.S. Pat. No. 5,159,135 (cotton); U.S. Pat. No. 5,824,877 (soybean); U.S. Pat. No. 5,463,174 (canola); U.S. Pat. No. 5,591,616 (corn); U.S. Pat. No. 6,384,301 (soybean); U.S. Pat. No. 7,026,528 (wheat) and U.S. Pat. No. 6,329,571 (rice), all of which are incorporated herein by reference. For Agrobacterium tumefaciens based plant transformation system, additional elements present on transformation constructs will include T-DNA left and right border sequences to facilitate incorporation of the recombinant polynucleotide into the plant genome.

In general it is useful to introduce recombinant DNA randomly, i.e. at a non-specific location, in the genome of a target plant line. In special cases it may be useful to target recombinant DNA insertion in order to achieve site-specific integration, for example, to replace an existing gene in the genome, to use an existing promoter in the plant genome, or to insert a recombinant polynucleotide at a predetermined site known to be active for gene expression. Several site specific recombination systems exist which are known to function implants include cre-lox as disclosed in U.S. Pat. No. 4,959,317 and FLP-FRT as disclosed in U.S. Pat. No. 5,527,695, both incorporated herein by reference.

Transformation methods of this invention are preferably practiced in tissue culture on media and in a controlled environment. “Media” refers to the numerous nutrient mixtures that are used to grow cells in vitro, that is, outside of the intact living organism. Recipient cell targets include, but are not limited to, meristem cells, callus, immature embryos and gametic cells such as microspores, pollen, sperm and egg cells. It is contemplated that any cell from which a fertile plant may be regenerated is useful as a recipient cell. Callus may be initiated from tissue sources including, but not limited to, immature embryos, seedling apical meristems, microspores and the like. Cells capable of proliferating as callus are also recipient cells for genetic transformation. Practical transformation methods and materials for making transgenic plants of this invention, for example various media and recipient target cells, transformation of immature embryo cells and subsequent regeneration of fertile transgenic plants are disclosed in U.S. Pat. Nos. 6,194,636 and 6,232,526, which are incorporated herein by reference.

The seeds of transgenic plants can be harvested from fertile transgenic plants and be used to grow progeny generations of transformed plants of this invention including hybrid plant lines for selection of plants having an enhanced trait. In addition to direct transformation of a plant with a recombinant DNA; transgenic plants can be prepared by crossing a first plant having a recombinant DNA with a second plant lacking the DNA. For example, recombinant DNA can be introduced into first plant line that is amenable to transformation to produce a transgenic plant which can be crossed with a second plant line to introgress the recombinant DNA into the second plant line. A transgenic plant with recombinant DNA providing an enhanced trait, e.g. enhanced yield, can be crossed with transgenic plant line having other recombinant DNA that confers another trait, for example herbicide resistance or pest resistance, to produce progeny plants having recombinant DNA that confers both traits. Typically, in such breeding for combining traits the transgenic plant donating the additional trait is a male line and the transgenic plant carrying the base traits is the female line. The progeny of this cross will segregate such that some of the plants will carry the DNA for both parental traits and some will carry DNA for one parental trait; such plants can be identified by markers associated with parental recombinant DNA, e.g. marker identification by analysis for recombinant DNA or, in the case where a selectable marker is linked to the recombinant, by application of the selecting agent such as a herbicide for use with a herbicide tolerance marker, or by selection for the enhanced trait. Progeny plants carrying DNA for both parental traits can be crossed back into the female parent line multiple times, for example usually 6 to 8 generations, to produce a progeny plant with substantially the same genotype as one original transgenic parental line but for the recombinant DNA of the other transgenic parental line

In the practice of transformation DNA is typically introduced into only a small percentage of target plant cells in any one transformation experiment. Marker genes are used to provide an efficient system for identification of those cells that are stably transformed by receiving and integrating a transgenic DNA construct into their genomes. Preferred marker genes provide selective markers which confer resistance to a selective agent, such as an antibiotic or herbicide. Any of the herbicides to which plants of this invention may be resistant are useful agents for selective markers. Potentially transformed cells are exposed to the selective agent. In the population of surviving cells will be those cells where, generally, the resistance-conferring gene is integrated and expressed at sufficient levels to permit cell survival. Cells may be tested further to confirm stable integration of the exogenous DNA. Commonly used selective marker genes include those conferring resistance to antibiotics such as kanamycin and paromomycin (nptH), hygromycin B (aph IV) and gentamycin (aac3 and aacC4) or resistance to herbicides such as glufosinate (bar or pat) and glyphosate (aroA or EPSPS). Examples of such selectable markers are illustrated in U.S. Pat. Nos. 5,550,318; 5,633,435; 5,780,708 and 6,118,047, all of which are incorporated herein by reference. Selectable markers which provide an ability to visually identify transformants can also be employed, for example, a gene expressing a colored or fluorescent protein such as a luciferase or green fluorescent protein (GFP) or a gene expressing a beta-glucuronidase or uidA gene (GUS) for which various chromogenic substrates are known.

Plant cells that survive exposure to the selective agent, or plant cells that have been scored positive in a screening assay, may be cultured in regeneration media and allowed to mature into plants. Developing plantlets regenerated from transformed plant cells can be transferred to plant growth mix, and hardened off, for example, in an environmentally controlled chamber at about 85% relative humidity, 600 ppm CO₂, and 25-250 microeinsteins m⁻² s⁻¹ of light, prior to transfer to a greenhouse or growth chamber for maturation. Plants are regenerated from about 6 weeks to 10 months after a transformant is identified, depending on the initial tissue. Plants may be pollinated using conventional plant breeding methods known to those of skill in the art and seed produced, for example self-pollination is commonly used with transgenic corn. The regenerated transformed plant or its progeny seed or plants can be tested for expression of the recombinant DNA and selected for the presence of enhanced agronomic trait.

### Transgenic Plants and Seeds

Transgenic plants derived from the plant cells of this invention are grown to generate transgenic plants having an enhanced trait as compared to a control plant and produce transgenic seed and pollen of this invention. Such plants with enhanced traits are identified by selection of transformed plants or progeny seed for the enhanced trait. For efficiency a selection method is designed to evaluate multiple transgenic plants (events) comprising the recombinant DNA, for example multiple plants from 2 to 20 or more transgenic events. Transgenic plants grown from transgenic seed provided herein demonstrate improved agronomic traits that contribute to increased yield or other trait that provides increased plant value, including, for example, improved seed quality. Of particular interest are plants having enhanced water use efficiency, enhanced cold tolerance, increased yield, enhanced nitrogen use efficiency, enhanced seed protein and enhanced seed oil.

Table 1 provides a list of protein encoding DNA (“genes”) that are useful as recombinant DNA for production of transgenic plants with enhanced agronomic trait, the elements of Table 1 are described by reference to:

“PEP SEQ ID NO” identifies an amino acid sequence from SEQ ID NO: 96 to 193.
“NUC SEQ ID NO” identifies a DNA sequence from SEQ ID NO: 1 to 95.
“BV id” is a reference to the identifying number in Table 4 of base vectors used for construction of the transformation vectors of the recombinant DNA. Construction of plant transformation constructs is illustrated in Example 1.
“Gene Name” is a common name for protein encoded by the recombinant DNA.
“Annotation” refers to a description of the top hit protein obtained from an amino acid sequence query of each PEP SEQ ID NO to GenBank database of the National Center for Biotechnology Information (ncbi). More particularly, “gi” is the GenBank ID number for the top BLAST hit;
“Description” refers to the description of the top BLAST hit;
“% id” refers to the percentage of identically matched amino acid residues alone the length of the portion of the sequences which is aliened by BLAST (−F T) between the sequence of interest provided herein and the hit sequence in GenBank.

TABLE 1

| Nuc | Pep | | | | | | |
|---|---|---|---|---|---|---|---|
| seq | seq | | | | Annotation | | |
| ID | ID | | BV | | % | GenBank | |
| NO | NO | Gene ID | ID | Gene Name | ID | ID | Desciption |
| 1 | 96 | PHE0004633_5508 | 4 | corn putative | 68 | 50947455 | ref\|XP_483255.1\|putative |
| | | | | transcription factor | | | transcription factor RAU1 |
| | | | | RAU1 | | | [Oryza sativa (japonica |
| | | | | | | | cultivar-group)] |
| 2 | 97 | PHE0004738_5674 | 1 | rice NAM protein | 82 | 55771315 | dbj\|BAD72224.1\|unknown |
| | | | | | | | protein [Oryza sativa |
| | | | | | | | (japonica cultivar-group)] |
| 3 | 98 | PHE0004814_5801 | 13 | corn KNOX family | 75 | 51535639 | dbj\|BAD37613.1\|KNOX |
| | | | | | | | family class 2 |
| | | | | | | | homeodomain protein |
| | | | | | | | [Oryza sativa (japonica |
| | | | | | | | cultivar-group)] |
| 4 | 99 | PHE0004817_5809 | 4 | corn PIF3-like | 69 | 50928761 | ref\|XP_473908.1\|OSJNBa0058K23.6 |
| | | | | family | | | [Oryza sativa |
| | | | | | | | (japonica cultivar-group)] |
| 5 | 100 | PHE0004817_5810 | 13 | corn PIF3-like | 69 | 50928761 | ref\|XP_473908.1\|OSJNBa0058K23.6 |
| | | | | family gene | | | [Oryza sativa |
| | | | | | | | (japonica cultivar-group)] |
| 6 | 101 | PHE0004821_5819 | 12 | corn PIF3-like | 61 | 55296133 | dbj\|BAD67851.1\|basic |
| | | | | family gene | | | helix-loop-helix protein |
| | | | | | | | SPATULA-like [Oryza |
| | | | | | | | sativa (japonica cultivar- |
| | | | | | | | group)] |
| 7 | 102 | PHE0004828_5826 | 4 | soy PIF3-like | 53 | 92897142 | gb\|ABE93546.1\|Helix-loop- |
| | | | | family gene | | | helix DNA-binding |
| | | | | | | | [Medicago truncatula] |
| 8 | 103 | PHE0004817_5901 | 12 | corn PIF3-like | 69 | 50928761 | ref\|XP_473908.1\|OSJNBa0058K23.6 |
| | | | | family gene | | | [Oryza sativa |
| | | | | | | | (japonica cultivar-group)] |
| 9 | 104 | PHE0004861_5910 | 4 | rice putative | 87 | 34899818 | ref\|NP_911255.1\|putative |
| | | | | SCARECROW | | | SCARECROW protein |
| | | | | protein | | | [Oryza sativa (japonica |
| | | | | | | | cultivar-group)] |
| 10 | 105 | PHE0004863_5912 | 4 | corn putative AT- | 61 | 50916020 | ref\|XP_468474.1\|putative |
| | | | | hook protein | | | AT-hook protein 1 [Oryza |
| | | | | | | | sativa (japonica cultivar- |
| | | | | | | | group)] |
| 11 | 106 | PHE0002062_5913 | 4 | corn R2R3 Myb | 59 | 50946113 | ref\|XP_482584.1\|putative |
| | | | | protein | | | typical P-type R2R3 Myb |
| | | | | | | | protein [Oryza sativa |
| | | | | | | | (japonica cultivar-group)] |
| 12 | 107 | PHE0002531_5926 | 17 | corn DNA-binding | 78 | 95102176 | gb\|ABF51012.1\|DOF1 [Zea |
| | | | | protein MNB1a | | | mays] |
| 13 | 108 | PHE0004914_5971 | 4 | soy syringolide- | 93 | 19911577 | dbj\|BAB86892.1\|syringolide- |
| | | | | induced protein | | | induced protein 1-3-1A |
| | | | | | | | [Glycine max] |
| 14 | 109 | PHE0004924_5982 | 4 | soy TCP family | 74 | 18396089 | ref\|NP_566164.1\|PTF1 |
| | | | | transcription factor | | | Plastid Transcription Factor |
| | | | | | | | 1 [Arabidopsis thaliana] |
| | | | | | | | gb\|AAM62743.1\|unknown |
| | | | | | | | [Arabidopsis thaliana] |
| 15 | 110 | PHE0004925_5983 | 4 | Arabidopsis | 94 | 15230904 | ref\|NP_191351.1\|DNA |
| | | | | squamosa | | | binding/transcription factor |
| | | | | promoter-binding | | | [Arabidopsis thaliana] |
| | | | | protein | | | sp\|Q9M2Q6\|SPL15_ARATH |
| | | | | | | | Squamosa promoter - |
| | | | | | | | binding-like protein 15 |
| 16 | 111 | PHE0004938_5994 | 4 | Arabidopsis | 96 | 15228553 | ref\|NP_186995.1\|RGL2 |
| | | | | gibberellin- | | | (RGA-LIKE 2); |
| | | | | responsive | | | transcription factor |
| | | | | modulator | | | [Arabidopsis thaliana] |
| | | | | | | | sp\|Q8GXW1\|RGL2_ARATH |
| | | | | | | | DELLA protein RGL2 |
| | | | | | | | (RGA-like protein 2) |
| | | | | | | | (Scarecrow-like protein 19) |
| 17 | 112 | PHE0004957_6019 | 4 | corn C2H2-type | 68 | 50933653 | ref\|XP_476354.1\|C2H2-type |
| | | | | zinc finger protein | | | zinc finger protein-like |
| | | | | | | | protein [Oryza sativa |
| | | | | | | | (japonica cultivar-group)] |
| 18 | 113 | PHE0004958_6020 | 4 | corn putative | 50 | 50940107 | ref\|XP_479581.1\|putative |
| | | | | ascorbate oxidase | | | ascorbate oxidase promoter- |
| | | | | promoter-binding | | | binding protein AOBP |
| | | | | protein | | | [Oryza sativa (japonica |
| | | | | | | | cultivar-group)] |
| | | | | | | | ref\|XP_506580.1\| |
| | | | | | | | PREDICTED |
| | | | | | | | OSJNBa0060O17.31 gene |
| | | | | | | | product [Oryza sativa |
| | | | | | | | (japonica cultivar-group)] |
| 19 | 114 | PHE0004959_6021 | 4 | corn putative | 41 | 56567581 | gb\|AAV98700.1\|BTH- |
| | | | | EREBP-type | | | induced ERF transcriptional |
| | | | | transcription factor | | | factor 1 [Oryza sativa |
| | | | | | | | (indica cultivar-group)] |
| 20 | 115 | PHE0004974_6040 | 4 | corn auxin | 74 | 108864436 | gb\|ABG22499.1\|Auxin |
| | | | | response factor | | | response factor 2, [Oryza |
| | | | | | | | sativa (japonica cultivar- |
| | | | | | | | group)] |
| 21 | 116 | PHE0004975_6041 | 4 | corn auxin | 67 | 77555450 | gb\|ABA98246.1\|Auxin |
| | | | | response factor | | | response factor 2, [Oryza |
| | | | | | | | sativa (japonica cultivar- |
| | | | | | | | group)] |
| 22 | 117 | PHE0004987_6056 | 4 | soy transfactor-like | 63 | 77403669 | dbj\|BAE46413.1\|MYB-CC |
| | | | | protein | | | type transfactor [Solanum |
| | | | | | | | tuberosum] |
| 23 | 118 | PHE0005005_7034 | 4 | corn putative Myb- | 52 | 37536868 | ref\|NP_922736.1\|putative |
| | | | | related protein | | | Myb-related protein [Oryza |
| | | | | | | | sativa (japonica cultivar- |
| | | | | | | | group)] |
| 24 | 119 | PHE0004877_7030 | 12 | corn response | 76 | 56784051 | dbj\|BAD82798.1\|putative |
| | | | | regulator ARR11 | | | response regulator 11 |
| | | | | | | | [Oryza sativa (japonica |
| | | | | | | | cultivar-group)] |
| 25 | 120 | PHE0006057_7048 | 13 | wheat PIF3-like | 40 | 109134123 | dbj\|BAC41905.1\|putative |
| | | | | family gene | | | bHLH transcription factor |
| | | | | | | | bHLH016 [Arabidopsis |
| | | | | | | | thaliana] |
| 26 | 121 | PHE0006057_7053 | 12 | wheat PIF3-like | 40 | 109134123 | dbj\|BAC41905.1\|putative |
| | | | | family gene | | | bHLH transcription factor |
| | | | | | | | bHLH016 [Arabidopsis |
| | | | | | | | thaliana] |
| 27 | 122 | PHE0006070_7067 | 4 | corn putative | 64 | 54291039 | dbj\|BAD61716.1\|transcription |
| | | | | transcription factor | | | factor-like [Oryza sativa |
| | | | | | | | (japonica cultivar-group)] |
| 28 | 123 | PHE0006073_7072 | 4 | corn putative bZIP | 64 | 54291039 | dbj\|BAD61716.1\|transcription |
| | | | | transcription factor | | | factor-like [Oryza sativa |
| | | | | | | | (japonica cultivar-group)] |
| 29 | 124 | PHE0006004_7082 | 4 | soy NAM like | 51 | 15224202 | gb\|AAD22369.1\|NAM (no |
| | | | | protein | | | apical meristem)-like |
| | | | | | | | protein [Arabidopsis |
| | | | | | | | thaliana] |
| | | | | | | | sp\|Q9SK55\|NAC42_ARATH |
| | | | | | | | Putative NAC domain- |
| | | | | | | | containing protein 42 |
| | | | | | | | (ANAC042) |
| 30 | 125 | PHE0006022_7105 | 4 | soy transcription | 82 | 18643341 | gb\|AAL76272.1\|transcription |
| | | | | factor EIL1 | | | factor EIL1 [Vigna |
| | | | | | | | radiata] |
| 31 | 126 | PHE0006023_7240 | 4 | Arabidopsis bHLH | 92 | 15241896 | ref\|NP_201067.1\|DNA |
| | | | | family protein | | | binding/transcription factor |
| | | | | | | | [Arabidopsis thaliana] |
| 32 | 127 | PHE0006191_7251 | 8 | EEM7 | 60 | 50936701 | ref\|XP_477878.1\|hypothetical |
| | | | | | | | protein [Oryza sativa |
| | | | | | | | (japonica cultivar-group)] |
| 33 | 128 | PHE0006237_7261 | 18 | Lycopersicon | 86 | 18650662 | gb\|AAL75809.1\|ethylene |
| | | | | SHN1 | | | response factor 1 |
| | | | | | | | [Lycopersicon esculentum] |
| 34 | 129 | PHE0006237_7274 | 17 | Lycopersicon | 86 | 18650662 | gb\|AAL75809.1\|ethylene |
| | | | | SHN1 | | | response factor 1 |
| | | | | | | | [Lycopersicon esculentum] |
| 35 | 130 | PHE0006237_7268 | 6 | Lycopersicon | 86 | 18650662 | gb\|AAL75809.1\|ethylene |
| | | | | SHN1 | | | response factor 1 |
| | | | | | | | [Lycopersicon esculentum] |
| 36 | 131 | PHE0006237_7277 | 5 | Lycopersicon | 86 | 18650662 | gb\|AAL75809.1\|ethylene |
| | | | | SHN1 | | | response factor 1 |
| | | | | | | | [Lycopersicon esculentum] |
| 37 | 132 | PHE0006237_7284 | 11 | Lycopersicon | 86 | 18650662 | gb\|AAL75809.1\|ethylene |
| | | | | SHN1 | | | response factor 1 |
| | | | | | | | [Lycopersicon esculentum] |
| 38 | 133 | PHE0004816_7303 | 4 | corn PIF3-like | 82 | 50928761 | ref\|XP_473908.1\|OSJNBa0058K23.6 |
| | | | | family gene | | | [Oryza sativa |
| | | | | | | | (japonica cultivar-group)] |
| 39 | 134 | PHE0006291_7319 | 17 | soy putative | 42 | 15227152 | ref\|NP_182310.1\|transcription |
| | | | | CONSTANS-like | | | factor/zinc ion binding |
| | | | | B-box zinc finger | | | [Arabidopsis thaliana] |
| | | | | protein | | | |
| 40 | 135 | PHE0004816_7421 | 12 | corn PIF3-like | 82 | 50928761 | ref\|XP_473908.1\|OSJNBa0058K23.6 |
| | | | | family gene | | | [Oryza sativa |
| | | | | | | | (japonica cultivar-group)] |
| 41 | 136 | PHE0004816_7418 | 4 | corn PIF3-like | 82 | 50928761 | ref\|XP_473908.1\|OSJNBa0058K23.6 |
| | | | | family gene | | | [Oryza sativa |
| | | | | | | | (japonica cultivar-group)] |
| 42 | 137 | PHE0003673_7430 | 4 | corn response | 76 | 56784051 | dbj\|BAD82798.1\|putative |
| | | | | regulator like | | | response regulator 11 |
| | | | | | | | [Oryza sativa (japonica |
| | | | | | | | cultivar-group)] |
| 43 | 138 | PHE0003664_7436 | 4 | soy AP2/EREBP | 48 | 15227980 | gb\|AAT44934.1\|putative |
| | | | | transcription factor | | | AP2/EREBP transcription |
| | | | | like | | | factor [Arabidopsis thaliana] |
| 44 | 139 | PHE0004816_7445 | 13 | corn PIF3-like | 82 | 50928761 | ref\|XP_473908.1\|OSJNBa0058K23.6 |
| | | | | family gene | | | [Oryza sativa |
| | | | | | | | (japonica cultivar-group)] |
| 45 | 140 | PHE0002149_7487 | 4 | corn DNA-binding | 66 | 50941323 | ref\|XP_480189.1\|putative |
| | | | | protein | | | LHY protein [Oryza sativa |
| | | | | | | | (japonica cultivar-group)] |
| 46 | 141 | PHE0006290_7498 | 4 | corn putative | 70 | 50912285 | ref\|XP_467550.1\|zinc-finger |
| | | | | CONSTANS-like | | | protein [Oryza sativa |
| | | | | B-box zinc finger | | | (japonica cultivar-group)] |
| | | | | protein | | | |
| 47 | 142 | PHE0006423_7664 | 4 | soy Myb61 | 56 | 92873337 | gb\|ABE81808.1\|Homeodomain- |
| | | | | | | | related [Medicago |
| | | | | | | | truncatula] |
| 48 | 143 | PHE0006384_7737 | 9 | rice R2R3 Myb | 55 | 50946113 | ref\|XP_482584.1\|putative |
| | | | | protein | | | typical P-type R2R3 Myb |
| | | | | | | | protein [Oryza sativa |
| | | | | | | | (japonica cultivar-group)] |
| 49 | 144 | PHE0006384_7789 | 13 | rice R2R3 Myb | 55 | 50946113 | ref\|XP_482584.1\|putative |
| | | | | protein | | | typical P-type R2R3 Myb |
| | | | | | | | protein [Oryza sativa |
| | | | | | | | (japonica cultivar-group)] |
| 50 | 145 | PHE0006507_7828 | 17 | Corn NFB1_23C | 98 | 50916531 | gb\|ABF96585.1\|CCAAT- |
| | | | | | | | binding transcription factor |
| | | | | | | | subunit A [Oryza sativa |
| | | | | | | | (japonica cultivar-group)] |
| 51 | 146 | PHE0006509_7846 | 19 | Arabidopsis | 60 | 62856979 | gb\|AAY16440.1\|squamosa |
| | | | | Gm2010 | | | promoter binding-like |
| | | | | | | | protein [Betula platyphylla] |
| 52 | 147 | PHE0006384_7839 | 19 | rice R2R3 Myb | 55 | 50946113 | ref\|XP_482584.1\|putative |
| | | | | protein | | | typical P-type R2R3 Myb |
| | | | | | | | protein [Oryza sativa |
| | | | | | | | (japonica cultivar-group)] |
| 53 | 148 | PHE0006448_7859 | 17 | Arabidopsis | 99 | 15217662 | ref\|NP_176634.1\|transcription |
| | | | | transcription factor | | | factor [Arabidopsis |
| | | | | | | | thaliana] gb\|AAN41333.1\| |
| | | | | | | | unknown protein |
| | | | | | | | [Arabidopsis thaliana] |
| 54 | 149 | PHE0006504_7876 | 17 | maize tubby 4 | 69 | 55733806 | gb\|AAV59313.1\|putative |
| | | | | | | | tubby protein [Oryza sativa |
| | | | | | | | (japonica cultivar-group)] |
| 55 | 150 | PHE0006057_7929 | 15 | wheat P1F3-like | 40 | 109134123 | dbj\|BAC41905.1\|putative |
| | | | | family gene | | | bHLH transcription factor |
| | | | | | | | bHLH016 [Arabidopsis |
| | | | | | | | thaliana] |
| 56 | 151 | PHE0003473_7927 | 9 | soy Zinc finger | 50 | 87162706 | gb\|ABD28501.1\|Zinc finger, |
| | | | | protein like | | | C2H2-type [Medicago |
| | | | | | | | truncatula] |
| 57 | 152 | PHE0002531_7985 | 45 | corn DNA-binding | 78 | 95102176 | gb\|ABF51012.1\|DOF1 [Zea |
| | | | | protein MNB1a | | | mays] |
| 58 | 153 | PHE0004463_8059 | 15 | soy ethylene | 48 | 15238727 | ref\|NP_197901.1\|DNA |
| | | | | response factor | | | binding/transcription factor |
| | | | | | | | [Arabidopsis thaliana] |
| 59 | 154 | PHE0001067_8154 | 10 | Arabidopsis | 89 | 15237035 | ref\|NP_195280.1\|DNA |
| | | | | homeodomain | | | binding/transcription factor |
| | | | | transcription factor | | | [Arabidopsis thaliana] |
| | | | | | | | sp\|O81788\|WOX13_ARATH |
| | | | | | | | WUSCHEL-related |
| | | | | | | | homeobox 13 |
| 60 | 155 | PHE0006350_8201 | 15 | GIA/RGA-like | 46 | 63054405 | gb\|AAY28970.1\|GIA/RGA- |
| | | | | gibberellin | | | like gibberellin response |
| | | | | response modulator | | | modulator [Gossypium |
| | | | | | | | hirsutum] |
| 61 | 156 | PHE0006605_8233 | 17 | Arabidopsis Zinc | 74 | 15230393 | ref\|NP_190677.1\|transcription |
| | | | | finger (GATA | | | factor [Arabidopsis |
| | | | | type) family | | | thaliana] |
| | | | | protein | | | |
| 62 | 157 | PHE0006546_8310 | 8 | Response regulator 9 | 61 | 55771374 | dbj\|BAD72541.1\|putative |
| | | | | | | | response regulator 9 [Oryza |
| | | | | | | | sativa (japonica cultivar- |
| | | | | | | | group)] |
| 63 | 158 | PHE0006527_8369 | 17 | NFB1-Q185H | 97 | 50916531 | gb\|ABF96585.1\|CCAAT- |
| | | | | | | | binding transcription factor |
| | | | | | | | subunit A [Oryza sativa |
| | | | | | | | (japonica cultivar-group)] |
| 64 | 159 | PHE0004938_8370 | 17 | Arabidopsis | 96 | 15228553 | sp\|QSGXW1\|RGL2_ARATH |
| | | | | gibberellin- | | | DELLA protein RGL2 |
| | | | | responsive | | | (RGA-like protein 2) |
| | | | | modulator | | | (Scarecrow-like protein 19) |
| | | | | | | | [Arabidopsis thaliana] |
| 65 | 160 | PHE0006774_8489 | 15 | NFB2_E76R_S83R | 82 | 115840 | sp\|P25209\|NFYB_MaizeNuclear |
| | | | | | | | transcription factor Y |
| | | | | | | | subunit B (NF-YB) (CAAT- |
| | | | | | | | box DNA-binding protein |
| | | | | | | | subunit B) |
| 66 | 161 | PHE0006778_8503 | 15 | NFB2_149R_C73S_C89S | 82 | 115840 | sp\|P25209\|NFYB_MaizeNuclear |
| | | | | | | | transcription factor Y |
| | | | | | | | subunit B (NF-YB) (CAAT- |
| | | | | | | | box DNA-binding protein |
| | | | | | | | subunit B) |
| 67 | 162 | PHE0006780_8502 | 15 | NFB2_C73S_C89S_L102R | 82 | 115840 | sp\|P25209\|NFYB_Maize |
| | | | | | | | Nuclear transcription factor |
| | | | | | | | Y subunit B (NF-YB) |
| | | | | | | | (CAAT-box DNA-binding |
| | | | | | | | protein subunit B) |
| 68 | 163 | PHE0006752_8521 | 16 | wheat AP1 | 75 | 30721847 | gb\|AAP33790.1\|MADS-box |
| | | | | (VRN1) | | | protein TaVRT-1 |
| | | | | | | | gb\|AAW73225.1\|VRN-B1 |
| | | | | | | | [Triticum aestivum] |
| 69 | 164 | PHE0006779_8565 | 15 | corn | 82 | 115840 | sp\|P25209\|NFYB_MaizeNuclear |
| | | | | NFB2_C73R_C89S | | | transcription factor Y |
| | | | | | | | subunit B (NF-YB) (CAAT- |
| | | | | | | | box DNA-binding protein |
| | | | | | | | subunit B) |
| 70 | 165 | PHE0006781_8573 | 15 | corn | 82 | 115840 | sp\|P25209\|NFYB_MaizeNuclear |
| | | | | NFB2_149R_C73R_C89S_L102R | | | transcription factor Y |
| | | | | | | | subunit B (NF-YB) (CAAT- |
| | | | | | | | box DNA-binding protein |
| | | | | | | | subunit B) |
| 71 | 166 | PHE0003664_8637 | 15 | soy AP2/EREBP | 48 | 15227980 | gb\|AAT44934.1\|putative |
| | | | | transcription factor | | | AP2/EREBP transcription |
| | | | | | | | factor [Arabidopsis thaliana] |
| 72 | 167 | PHE0006004_8667 | 15 | soy NAM like | 51 | 15224202 | ref\|NP_181828.1\|ANAC042; |
| | | | | protein | | | transcription factor |
| | | | | | | | gb\|AAD22369.1\|NAM (no |
| | | | | | | | apical meristem)-like |
| | | | | | | | protein [Arabidopsis |
| | | | | | | | thaliana] |
| 73 | 168 | PHE0006022_8690 | 15 | soy transcription | 82 | 18643341 | gb\|AAL76272.1\|transcription |
| | | | | factor EIL1 | | | factor EIL1 [Vigna |
| | | | | | | | radiata] |
| 74 | 169 | PHE0006290_8689 | 15 | corn putative | 70 | 50912285 | ref\|XP_467550.1\|zinc-finger |
| | | | | CONSTANS-like | | | protein [Oryza sativa |
| | | | | B-box zinc finger | | | (japonica cultivar-group)] |
| | | | | protein | | | |
| 75 | 170 | PHE0006423_8696 | 15 | soy Myb61 | 56 | 92873337 | gb\|ABE81808.1\|Homeodomain- |
| | | | | | | | related [Medicago |
| | | | | | | | truncatula] |
| 76 | 171 | PHE0002149_8748 | 15 | corn DNA-binding | 66 | 50941323 | ref\|XP_480189.1\|putative |
| | | | | protein | | | LHY protein [Oryza sativa |
| | | | | | | | (japonica cultivar-group)] |
| 77 | 172 | PHE0006023_8762 | 15 | Arabidopsis bHLH | 92 | 15241896 | ref\|NP_201067.1\|DNA |
| | | | | family protein | | | binding/transcription factor |
| | | | | | | | [Arabidopsis thaliana] |
| 78 | 173 | PHE0004987_8771 | 15 | soy transfactor-like | 63 | 77403669 | dbj\|BAE46413.1\|MYB-CC |
| | | | | protein | | | type transfactor [Solanum |
| | | | | | | | tuberosum] |
| 79 | 174 | PHE0006858_8859 | 7 | corn MADS box | 97 | 939781 | gb\|AAB00079.1\|MADS box |
| | | | | protein | | | protein |
| 80 | 175 | PHE0006860_8863 | 7 | corn kernel specific | 100 | 939779 | gb\|AAB00078.1\|MADS box |
| | | | | MADS | | | protein |
| 81 | 176 | PHE0006955_9129 | 20 | Lycopersicon | 67 | 24967140 | gb\|AAM33103.2\|TAGL12 |
| | | | | esculentum | | | transcription factor |
| | | | | TAGL12 | | | [Lycopersicon esculentum] |
| | | | | transcription factor | | | |
| 82 | 177 | PHE0006951_9137 | 20 | AT5g52010/MSG15_9 | 97 | 15242250 | ref\|NP_200014.1\|nucleic |
| | | | | | | | acid binding/transcription |
| | | | | | | | factor/zinc ion binding |
| | | | | | | | [Arabidopsis thaliana] |
| 83 | 178 | PHE0006981_9158 | 15 | GRAS family | 59 | 92886232 | gb\|ABE88228.1\|GRAS |
| | | | | transcription factor | | | family transcription factor |
| | | | | | | | [Medicago truncatula] |
| 84 | 179 | PHE0006951_9173 | 15 | AT5g52010/MSG15_9 | 97 | 15242250 | ref\|NP_200014.1\|nucleic |
| | | | | | | | acid binding/transcription |
| | | | | | | | factor/zinc ion binding |
| | | | | | | | [Arabidopsis thaliana] |
| 85 | 180 | PHE0004646_PMON94356.pep | 17 | Arabidopsis NAM | 86 | 9758529 | dbj\|BAB08905.1\|unnamed |
| | | | | family protein | | | protein product |
| | | | | | | | [Arabidopsis thaliana] |
| 86 | 181 | PHE0004723_PMON94660.pep | 17 | soy auxin-induced | 92 | 114733 | sp\|P13088\|AUX22_SOYBN |
| | | | | protein | | | Auxin-induced protein |
| | | | | | | | AUX22 |
| 87 | 182 | PHE0004648_PMON95051.pep | 4 | Arabidopsis | 93 | 20152540 | emb\|CAD29662.1\|putative |
| | | | | putative auxin | | | auxin response factor 23 |
| | | | | response factor 23 | | | [Arabidopsis thaliana] |
| 88 | 183 | PHE0004357_PMON94163.pep | 2 | corn | 47 | 50927517 | ref\|XP_473403.1\| |
| | | | | OSJNBa0079A21.14 | | | OSJNBa0079A21.14 |
| | | | | | | | [Oryza sativa |
| | | | | | | | (japonica cultivar-group)] |
| 89 | 184 | PHE0004646_PMON94352.pep | 2 | Arabidopsis NAM | 86 | 9758529 | dbj\|BAB08905.1\|unnamed |
| | | | | family protein | | | protein product |
| | | | | | | | [Arabidopsis thaliana] |
| 90 | 185 | PHE0004624_PMON94400.pep | 2 | soy auxin response | 65 | 30027167 | gb\|AAP06759.1\|auxin |
| | | | | factor-like protein | | | response factor-like protein |
| | | | | | | | [Mangifera indica] |
| 91 | 186 | PHE0004463_PMON94432.pep | 2 | soy ethylene | 48 | 15238727 | ref\|NP_197901.1\|DNA |
| | | | | response factor | | | binding/transcription factor |
| | | | | | | | [Arabidopsis thaliana] |
| 92 | 187 | PHE0004356_PMON93862.pep | 1 | corn LEC2/FUS3 | 66 | 56785317 | dbj\|BAD82277.1\|regulatory |
| | | | | | | | protein Viviparous-1-like |
| | | | | | | | [Oryza sativa (japonica |
| | | | | | | | cultivar-group)] |
| 93 | 188 | PHE0004332_PMON95104.pep | 14 | tomato Pti4 | 85 | 3342211 | gb\|AAC50047.1\|Pti4 |
| | | | | | | | [Lycopersicon esculentum] |
| 94 | 189 | PHE0004644_PMON95096.pep | 3 | corn ICE1-like | 50 | 77551194 | gb\|ABA93991.1\|Helix-loop- |
| | | | | | | | helix DNA-binding domain |
| | | | | | | | containing protein [Oryza |
| | | | | | | | sativa (japonica cultivar- |
| | | | | | | | group)] |
| 95 | 190 | PHE0004723_PMON95121.pep | 4 | soy auxin-induced | 92 | 114733 | sp\|P13088\|AUX22_SOYBN |
| | | | | protein | | | Auxin-induced protein |
| | | | | | | | AUX22 |

Selection Methods for Transgenic Plants with Enhanced Agronomic Trait

Within a population of transgenic plants regenerated from plant cells transformed with the recombinant DNA construct many plants that survive to fertile transgenic plants that produce seeds and progeny plants will not exhibit an enhanced agronomic trait. Selection from the population is necessary to identify one or more transgenic plant cells that can provide plants with the enhanced trait. Transgenic plants having enhanced agronomic traits are selected from populations of plants regenerated or derived from plant cells transformed as described herein by evaluating the plants in a variety of assays to detect an enhanced trait, e.g. enhanced water use efficiency, enhanced cold tolerance, increased yield, enhanced nitrogen use efficiency, enhanced seed protein and enhanced seed oil. These assays also may take many forms including, but not limited to, direct screening for the trait in a greenhouse or field trial or by screening for a surrogate trait. Such analyses can be directed to detecting changes in the chemical composition, biomass, physiological properties, morphology of the plant. Changes in chemical compositions such as nutritional composition of grain can be detected by analysis of the seed composition and content of protein, free amino acids, oil, free fatty acids, starch or tocopherols. Changes in biomass characteristics can be made on greenhouse or field grown plants and can include plant height, stem diameter, root and shoot dry weights; and, for corn plants, ear length and diameter. Changes in physiological properties can be identified by evaluating responses to stress conditions, for example assays using imposed stress conditions such as water deficit, nitrogen deficiency, cold growing conditions, pathogen or insect attack or light deficiency, or increased plant density. Changes in morphology can be measured by visual observation of tendency of a transformed plant with an enhanced agronomic trait to also appear to be a normal plant as compared to changes toward bushy, taller, thicker, narrower leaves, striped leaves, knotted trait, chlorosis, albino, anthocyanin production, or altered tassels, ears or roots. Other selection properties include days to pollen shed, clays to silking, leaf extension rate, chlorophyll content, leaf temperature, stand, seedling vigor, internode length, plant height, leaf number, leaf area, tillering, brace roots, stay green, stalk lodging, root lodging, plant health, barreness/prolificacy, green snap, and pest resistance. In addition, phenotypic characteristics of harvested grain may be evaluated, including number of kernels per row on the ear, number of rows of kernels on the ear, kernel abortion, kernel weight, kernel size, kernel density and physical grain quality. Although the plant cells and methods of this invention can be applied to any plant cell, plant, seed or pollen, e.g. any fruit, vegetable, grass, tree or ornamental plant, the various aspects of the invention are preferably applied to corn, soybean, cotton, canola, alfalfa, wheat and rice plants. In many cases the invention is applied to corn plants that are inherently resistant to disease from the Mal de Rio Cuarto virus or the Puccina sorghi fungus or both.

The following examples are included to demonstrate aspects of the invention, those of skill in the art should, in light of the present disclosure, appreciate that many changes can be made in the specific aspects which are disclosed and still obtain a like or similar results without departing from the spirit and scope of the invention.

### Example 1

### Plant Expression Constructs

This example illustrates the construction of plasmids for transferring recombinant DNA into plant cells which can be regenerated into transgenic plants of this invention.

A. Plant Expression Constructs for Corn Transformation

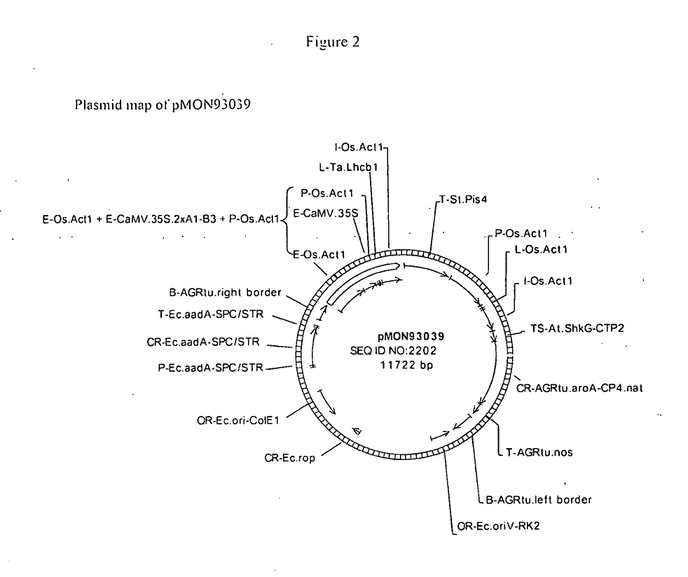

A base corn plant transformation vector pMON93039, as set forth in SEQ ID NO: 2202, illustrated in Table 2 and FIG. 2, was fabricated for use in preparing recombinant DNA for Agrobacterium-mediated transformation into corn tissue.

TABLE 2

| | | | Coordinates of |
|---|---|---|---|
| Function | Name | Annotation | SEQ ID NO: 2202 |
| Agro T-DNA | B-AGRtu.right border | Agro right border sequence, | 11364-11720 |
| transfer | | essential for transfer of T- | |
| | | DNA. | |
| Gene of | E-Os.Act1 | Upstream promoter region | 19-775 |
| interest | | of the rice actin 1 gene | |
| expression | E-CaMV.35S.2xA1-B3 | Duplicated 35S A1-B3 | 788-1120 |
| cassette | | domain without TATA box | |
| | P-Os.Act1 | Promoter region of the rice | 1125-1204 |
| | | actin 1 gene | |
| | L-Ta.Lhcb1 | 5′ untranslated leader of | 1210-1270 |
| | | wheat major chlorophyll | |
| | | a/b binding protein | |
| | I-Os.Act1 | First intron and flanking | 1287-1766 |
| | | UTR exon sequences from | |
| | | the rice actin 1 gene | |
| | T-St.Pis4 | 3′ non-translated region of | 1838-2780 |
| | | the potato proteinase | |
| | | inhibitor II gene which | |
| | | functions to direct | |
| | | polyadenylation of the | |
| | | mRNA | |
| Plant | P-Os.Act1 | Promoter from the rice | 2830-3670 |
| selectable | | actin 1 gene | |
| marker | L-Os.Act1 | First exon of the rice actin 1 | 3671-3750 |
| expression | | gene | |
| cassette | I-Os.Act1 | First intron and flanking | 3751-4228 |
| | | UTR exon sequences from | |
| | | the rice actin 1 gene | |
| | TS-At.ShkG-CTP2 | Transit peptide region of | 4238-4465 |
| | | Arabidopsis EPSPS | |
| | CR-AGRtu.aroA-CP4.nat | Coding region for bacterial | 4466-5833 |
| | | strain CP4 native aroA | |
| | | gene | |
| | T-AGRtu.nos | A 3′ non-translated region | 5849-6101 |
| | | of the nopaline synthase | |
| | | gene of Agrobacterium | |
| | | tumefaciens Ti plasmid | |
| | | which functions to direct | |
| | | polyadenylation of the | |
| | | mRNA. | |
| Agro T-DNA | B-AGRtu.left border | Agro left border sequence, | 6168-6609 |
| transfer | | essential for transfer of T- | |
| | | DNA. | |
| Maintenance | OR-Ec.oriV-RK2 | The vegetative origin of | 6696-7092 |
| in E. coli | | replication from plasmid | |
| | | RK2. | |
| | CR-Ec.rop | Coding region for repressor | 8601-8792 |
| | | of primer from the ColE1 | |
| | | plasmid. Expression of this | |
| | | gene product interferes with | |
| | | primer binding at the origin | |
| | | of replication, keeping | |
| | | plasmid copy number low. | |
| | OR-Ec.ori-ColE1 | The minimal origin of | 9220-9808 |
| | | replication from the E. coli | |
| | | plasmid ColE1. | |
| | P-Ec.aadA-SPC/STR | Promoter for Tn7 | 10339-10380 |
| | | adenylyltransferase (AAD | |
| | | (3″)) | |
| | CR-Ec.aadA-SPC/STR | Coding region for Tn7 | 10381-11169 |
| | | adenylyltransferase (AAD | |
| | | (3″)) conferring | |
| | | spectinomycin and | |
| | | streptomycin resistance. | |
| | T-Ec.aadA-SPC/STR | 3′ UTR from the Tn7 | 11170-11227 |
| | | adenylyltransferase (AAD | |
| | | (3″)) gene of E. coli. | |

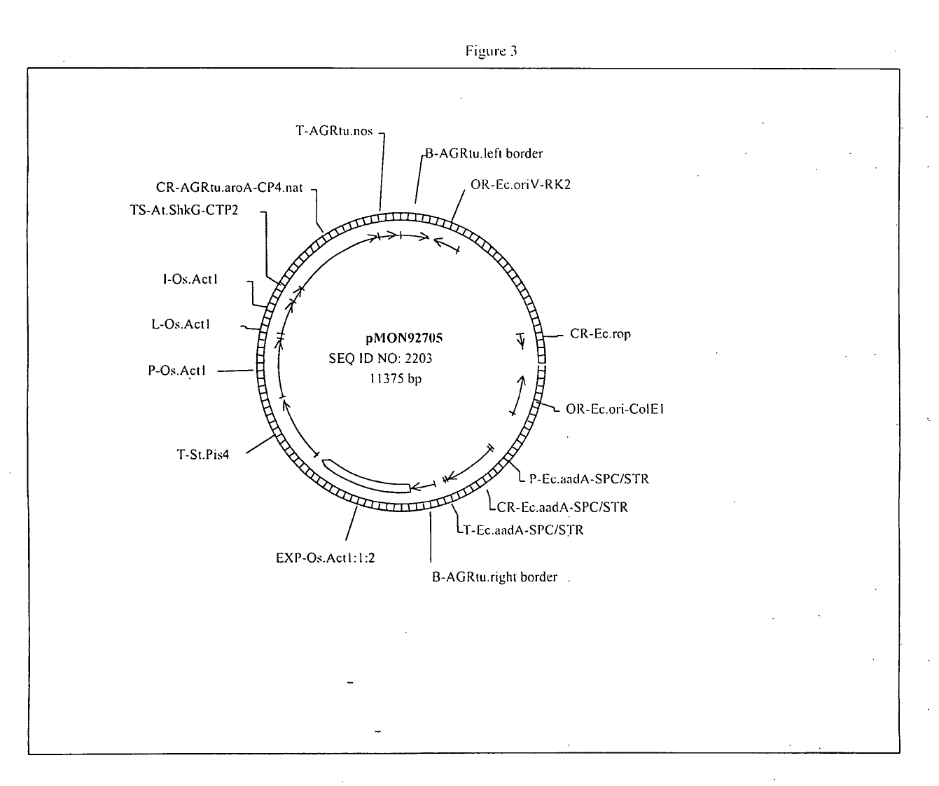

Another embodiment of corn plant transformation base vector is pMON92705, as set forth in SEQ ID NO: 2203, illustrated in Table 3 and FIG. 3, which was fabricated for use in preparing recombinant DNA for Agrobacterium-mediated transformation into corn tissue.

Other base vectors similar to those described above were also constructed as listed in Table 4. See Table 4 for a summary of base vector plasmids and base vector ID's which are referenced in Table 1. Also see Table 5 for a summary of regulatory elements used in the gene expression cassette for these base vectors and SEQ ID NOs for elements.

TABLE 3

| | | | Coordinates of |
|---|---|---|---|
| Function | Name | Annotation | SEQ ID NO: 2203 |
| Agro T-DNA | B-AGRtu. right border | Agro right border sequence, | 5206-5562 |
| transfer | | essential for transfer of T- | |
| | | DNA. | |
| Gene of | P-Os.Act1 | Promoter from rice actin 1 | 5580-6423 |
| interest | | gene | |
| expression | L-Os.Act1 | 5′ UTR of rice Act7 (or 1) | 6424-6503 |
| cassette | | gene | |
| | I-Os.Act1 | Intron from the rice actin7 | 6504-6980 |
| | | gene | |
| | T-St.Pis4 | 3′ non-translated region of the | 7055-7997 |
| | | potato proteinase inhibitor II | |
| | | gene which functions to direct | |
| | | polyadenylation of the mRNA | |
| Plant | P-Os.Act1 | Promoter from the rice actin 1 | 8047-8887 |
| selectable | | gene | |
| marker | L-Os.Act1 | First exon of the rice actin 1 | 8888-8967 |
| expression | | gene | |
| cassette | I-Os.Act1 | First intron and flanking UTR | 8968-9445 |
| | | exon sequences from the rice | |
| | | actin 1 gene | |
| | TS-At.ShkG-CTP2 | Transit peptide region of | 9455-9682 |
| | | Arabidopsis EPSPS | |
| | CR-AGRtu.aroA-CP4.nat | Coding region for bacterial | 9683-11050 |
| | | strain CP4 native aroA gene | |
| | T-AGRtu.nos | A 3′ non-translated region of | 11066-11318 |
| | | the nopaline synthase gene of | |
| | | Agrobacterium tumefaciens Ti | |
| | | plasmid which functions to | |
| | | direct polyadenylation of the | |
| | | mRNA. | |
| Agro T-DNA | B-AGRtu.left border | Agro left border sequence, | 10-451 |
| transfer | | essential for transfer of T- | |
| | | DNA. | |
| Maintenance | OR-Ec.oriV-RK2 | The vegetative origin of | 538-934 |
| in E. coli | | replication from plasmid RK2. | |
| | CR-Ec.rop | Coding region for repressor of | 2443-2634 |
| | | primer from the ColE1 | |
| | | plasmid. Expression of this | |
| | | gene product interferes with | |
| | | primer binding at the origin of | |
| | | replication, keeping plasmid | |
| | | copy number low. | |
| | OR-Ec.ori-ColE1 | The minimal origin of | 3062-3650 |
| | | replication from the E. coli | |
| | | plasmid ColE1. | |
| | P-Ec.aadA-SPC/STR | Promoter for Tn7 | 4181-4222 |
| | | adenylyltransferase (AAD | |
| | | (3″)) | |
| | CR-Ec.aadA-SPC/STR | Coding region for Tn7 | 4223-5011 |
| | | adenylyltransferase (AAD | |
| | | (3″)) conferring | |
| | | spectinomycin and | |
| | | streptomycin resistance. | |
| | T-Ec.aadA-SPC/STR | 3′ UTR from the Tn7 | 5012-5069 |
| | | adenylyltransferase (AAD | |
| | | (3″)) gene of E. coli. | |

Primers for PCR amplification of protein coding nucleotides of recombinant DNA are designed at or near the start and stop codons of the coding sequence, in order to eliminate most of the 5′ and 3′ untranslated regions. Each recombinant DNA coding for a protein identified in Table 1 is amplified by PCR prior to insertion into the insertion site within the gene of interest expression cassette of one of the base vectors as referenced in Table 1.

TABLE 4

| Base Vector ID | |
|---|---|
| | Base Vector for Corn |
| 1 | pMON74577 |
| 2 | pMON82060 |
| 3 | pMON82921 |
| 4 | pMON92705 |
| 5 | pMON92708 |
| 6 | pMON92712 |
| 7 | pMON92713 |
| 8 | pMON92715 |
| 9 | pMON92716 |
| 10 | pMON92718 |
| 11 | pMON92719 |
| 12 | pMON92722 |
| 13 | pMON92724 |
| 14 | pMON92725 |
| 15 | pMON93039 |
| 16 | pMON93043 |
| | Base Vector for |
| | Soybean And Canola |
| 17 | pMON82053 |
| 18 | pMON92669 |
| 19 | pMON92671 |
| 20 | pMON99006 |

TABLE 5

| | | SEQ ID | | SEQ ID | | SEQ ID |
|---|---|---|---|---|---|---|
| Vector | Promoter | NO | Leader | NO | Intron | NO |
| pMON74577 | P-Hv.Per1 | 2167 | L-Hv.Per1 | 2182 | I-Zm.DnaK | 2197 |
| pMON82053 | P-CaMV.35S-enh | 2168 | NONE | / | NONE | / |
| pMON82060 | P-Os.Act1 | 2169 | L-Os.Act1 | 2183 | I-Os.Act1 | 2198 |
| pMON82921 | P-Zm.Cik 1 | 2170 | L-Zm.Cik 1 | 2184 | I-Zm.Cik 1 | 2199 |
| pMON92705 | P-Os.Act1 | 2169 | L-Os.Act1 | 2183 | I-Os.Act1 | 2198 |
| pMON92708 | P-Zm.CA4H | 2171 | L-Zm.CA4H | 2185 | NONE | / |
| pMON92712 | P-Os.Cut1 | 2172 | L-Os.Cut1 | 2186 | I-Zm.DnaK | 2197 |
| pMON92713 | P-Zm.P39486 | 2173 | L-Zm.39486 | 2187 | I-Zm.DnaK | 2197 |
| pMON92715 | P-Hv.Per1 | 2167 | L-Hv.Per1 | 2182 | I-Zm.DnaK | 2197 |
| pMON92716 | P-Zm.FDA | 2174 | L-Zm.FDA | 2188 | I-Zm.DnaK | 2197 |
| pMON92718 | P-Zm.Cik 1 | 2170 | L-Zm.Cik1 | 2184 | I-Zm.Cik1 | 2199 |
| pMON92719 | P-Zm.RAB17 | 2175 | L- | 2189 | I-Zm.DnaK | 2197 |
| | | | Zm.RAB17 | | | |
| pMON92722 | P-CaMV.35S-enh | 2168 | L- | 2190 | I-Zm.DnaK | 2197 |
| | | | CaMV.35S | | | |
| pMON92724 | P-Zm.-636aldolase-0:1:2 + | 2176 | L-Zm.PPDK | 2191 | I-Zm.DnaK | 2197 |
| | P-Zm.PPDK | | | | | |
| pMON93039 | E-Os.Act1 + E- | 2177 | L-Ta.Lhcb1 | 2192 | I-Os.Act1 | 2198 |
| | CaMV.35S.2xA1-B3 + P- | | | | | |
| | Os.Act1 | | | | | |
| pMON93043 | P-Zm.EM | 2178 | L-Zm.EM | 2193 | I-Zm.DnaK | 2197 |
| pMON92669 | P-At.Rca | 2179 | L-At.Rca | 2194 | NONE | / |
| pMON92671 | P-At.SAMS3 | 2180 | L- | 2195 | I-At.SAMS3 | 2200 |
| | | | At.SAMS3 | | | |
| pMON99006 | P-CaMV.35S-enh | 2168 | NONE | / | NONE | / |
| pMON92725 | P-Zm.HRGP | 2181 | L-Zm.HRGP | 2196 | I-Zm.DnaK | 2197 |

B. Plasmids for Use in Transformation of Soybean and Canola are Also Prepared

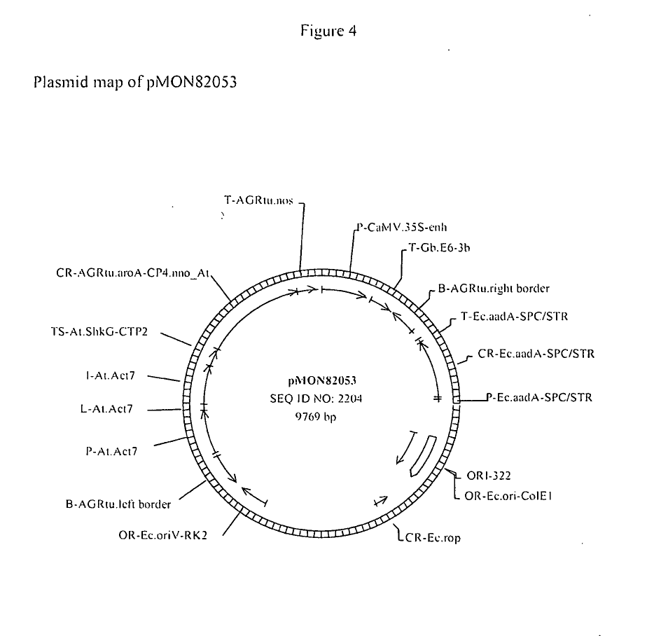

Elements of an exemplary common expression vector plasmid pMONS2053 are shown in Table 6 below and FIG. 4.

TABLE 6

| | | | Coordinates of |
|---|---|---|---|
| Function | Name | Annotation | SEQ ID NO: 2204 |
| Agro T-DNA | B-AGRtu.left | Agro left border sequence, essential for | 6144-6585 |
| transfer | border | transfer of T-DNA. | |
| Plant selectable | P-At.Act7 | Promoter from the Arabidopsis actin 7 | 6624-7861 |
| marker | | gene | |
| expression | | | |
| cassette | | | |
| | L-At.Act7 | 5′ UTR of Arabidopsis Act7 gene | |
| | I-At.Act7 | Intron from the Arabidopsis actin7 gene | |
| | TS-At.ShkG-CTP2 | Transit peptide region of Arabidopsis | 7864-8091 |
| | | EPSPS | |
| | CR-AGRtu.aroA- | Synthetic CP4 coding region with dicot | 8092-9459 |
| | CP4.nno_At | preferred codon usage. | |
| | T-AGRtu.nos | A 3′ non-translated region of the | 9466-9718 |
| | | nopaline synthase gene of | |
| | | Agrobacterium tumefaciens Ti plasmid | |
| | | which functions to direct | |
| | | polyadenylation of the mRNA. | |
| Gene of interest | P-CaMV.35S-enh | Promoter for 35S RNA from CaMV | 1-613 |
| expression | | containing a duplication of the −90 to −350 | |
| cassette | | region. | |
| | T-Gb.E6-3b | 3′ untranslated region from the liber | 688-1002 |
| | | protein E6 gene of sea-island cotton; | |
| Agro T-DNA | B-AGRtu.right | Agro right border sequence, essential | 1033-1389 |
| transfer | border | for transfer of T-DNA. | |
| Maintenance in | OR-Ec.oriV-RK2 | The vegetative origin of replication | 5661-6057 |
| E. coli | | from plasmid RK2. | |
| | CR-Ec.rop | Coding region for represser of primer | 3961-4152 |
| | | from the ColE1 plasmid. Expression of | |
| | | this gene product interferes with primer | |
| | | binding at the origin of replication, | |
| | | keeping plasmid copy number low. | |
| | OR-Ec.ori-ColE1 | The minimal origin of replication from | 2945-3533 |
| | | the E. coli plasmid ColE1. | |
| | P-Ec.aadA- | Promoter for Tn7 adenylyltransferase | 2373-2414 |
| | SPC/STR | (AAD(3″)) | |
| | CR-Ec.aadA- | Coding region for Tn7 | 1584-2372 |
| | SPC/STR | adenylyltransferase (AAD (3″)) | |
| | | conferring spectinomycin and | |
| | | streptomycin resistance. | |
| | T-Ec.aadA- | 3′ UTR from the Tn7 | 1526-1583 |
| | SPC/STR | adenylyltransferase (AAD (3″)) gene of | |
| | | E. coli. | |

Primers for PCR amplification of protein coding nucleotides of recombinant DNA are designed at or near the start and stop codons of the coding sequence, in order to eliminate most of the 5′ and 3′ untranslated regions. Each recombinant DNA coding for a protein identified in Table 1 is amplified by PCR prior to insertion into the insertion site within the gene of interest expression cassette of one of the base vectors as referenced in Table 1.

C. Cotton Transformation Vector

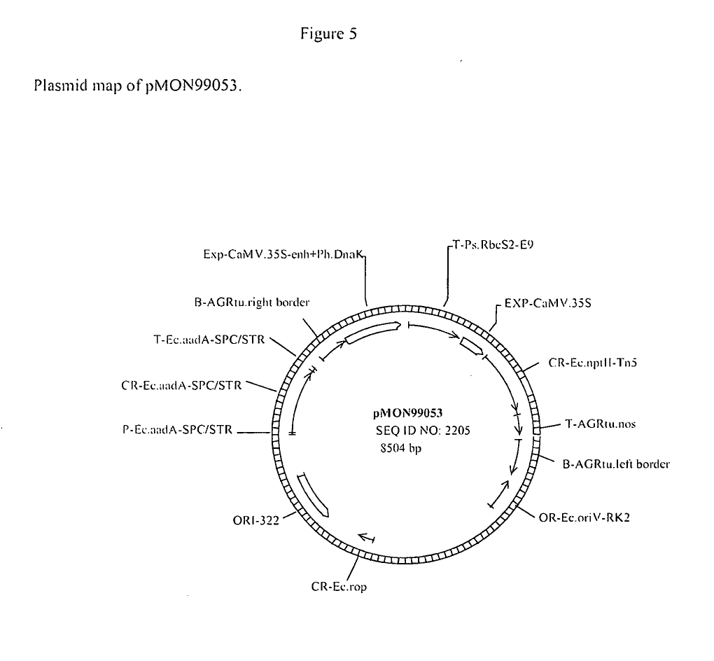

Recombinant DNA constructs for use in transformation of cotton are also prepared. Elements of an exemplary common expression vector plasmid pMON99053 are shown in Table 7 below and FIG. 5. Primers for PCR amplification of protein coding nucleotides of recombinant DNA are designed at or near the start and stop codons of the coding sequence, in order to eliminate most of the 5′ and 3′ untranslated regions. Each recombinant DNA coding for a protein identified in Table 1 is amplified by PCR prior to insertion into the insertion site within the gene of interest expression cassette of the base vector in Table 7.

TABLE 7

| | | | Coordinates of |
|---|---|---|---|
| Function | Name | Annotation | SEQ ID NO: 2205 |
| Agrobacterium | B-AGRtu.right border | Agro right border sequence, | 1-357 |
| T-DNA transfer | | essential for transfer of T- | |
| | | DNA. | |
| Gene of interest | Exp-CaMV.35S-enh + Ph.DnaK | Enhanced version of the 35S | 388-1091 |
| expression | | RNA promoter from CaMV | |
| cassette | | plus the petunia hsp70 5′ | |
| | | untranslated region | |
| | T-Ps.RbcS2-E9 | The 3′ non-translated region of | 1165-1797 |
| | | the pea RbcS2 gene which | |
| | | functions to direct | |
| | | polyadenylation of the mRNA. | |
| Plant selectable | Exp-CaMV.35S | Promoter and 5′ untranslated | 1828-2151 |
| marker | | region from the 35S RNA of | |
| expression | | CaMV | |
| cassette | CR-Ec.nptII-Tn5 | Coding region for neomycin | 2185-2979 |
| | | phosphotransferase gene from | |
| | | transposon Tn5 which confers | |
| | | resistance to neomycin and | |
| | | kanamycin. | |
| | T-AGRtu.nos | A 3′ non-translated region of | 3011-3263 |
| | | the nopaline synthase gene of | |
| | | Agrobacterium tumefaciens Ti | |
| | | plasmid which functions to | |
| | | direct polyadenylation of the | |
| | | mRNA. | |
| Agrobacterium | B-AGRtu.left border | Agro left border sequence, | 3309-3750 |
| T-DNA transfer | | essential for transfer of T- | |
| | | DNA. | |
| Maintenance in | OR-Ec.oriV-RK2 | The vegetative origin of | 3837-4233 |
| E. coli | | replication from plasmid RK2. | |
| | CR-Ec.rop | Coding region for repressor of | 5742-5933 |
| | | primer from the ColE1 | |
| | | plasmid. Expression of this | |
| | | gene product interferes with | |
| | | primer binding at the origin of | |
| | | replication, keeping plasmid | |
| | | copy number low. | |
| | OR-Ec.ori-ColEI | The minimal origin of | 6361-6949 |
| | | replication from the E. coli | |
| | | plasmid ColE1. | |
| | P-Ec.aadA-SPC/STR | Promoter for Tn7 | 7480-7521 |
| | | adenylyltransferase | |
| | | (AAD(3″)) | |
| | CR-Ec.aadA-SPC/STR | Coding region for Tn7 | 7522-8310 |
| | | adenylyltransferase | |
| | | (AAD(3″)) conferring | |
| | | spectinomycin and | |
| | | streptomycin resistance. | |
| | T-Ec.aadA-SPC/STR | 3′ UTR from the Tn7 | 8311-8368 |
| | | adenylyltransferase | |
| | | (AAD(3″)) gene of E. coli. | |

### Example 2

### Corn Transformation

This example illustrates the production and identification of transgenic corn cells in seed of transgenic corn plants having an enhanced agronomic trait, i.e. enhanced nitrogen use efficiency, increased yield, enhanced water use efficiency, enhanced tolerance to cold and/or improved seed compositions as compared to control plants. Transgenic corn cells are prepared with recombinant DNA construct expressing each of the protein encoding DNAs listed in Table 1 by Agrobacterium-mediated transformation using the corn transformation vectors as disclosed in Example 1. Corn transformation is effected using methods disclosed in U.S. Patent Application Publication 2004/0344075 A1 where corn embryos are inoculated and co-cultured with the Agrobacterium tumefaciens strain ABI and the corn transformation vector. To regenerate transgenic corn plants the transgenic callus resulting from transformation is placed on media to initiate shoot development in plantlets which are transferred to potting soil for initial growth in a growth chamber followed by a mist bench before transplanting to pots where plants are grown to maturity. The plants are self fertilized and seed is harvested for screening as seed, seedlings or progeny R2 plants or hybrids, e.g., for yield trials in the screens indicated above.

Many transgenic events which survive to fertile transgenic plants that produce seeds and progeny plants do not exhibit an enhanced agronomic trait. The transgenic plants and seeds having the transgenic cells of this invention which have recombinant DNA imparting the enhanced agronomic traits are identified by screening for nitrogen use efficiency, yield, water use efficiency, cold tolerance and improved seed composition as reported in Example 7.

### Example 3

### Soybean Transformation

This example illustrates the production and identification of transgenic soybean cells in seed of transgenic soybean plants having an enhanced agronomic trait, i.e. enhanced nitrogen use efficiency, increased yield, enhanced water use efficiency, enhanced tolerance to cold and/or improved seed compositions as compared to control plants. Transgenic soybean cells are prepared with recombinant DNA expressing each of the protein encoding DNAs listed in Table 1 by Agrobacterium-mediated transformation using the soybean transformation vectors disclosed in Example 1. Soybean transformation is effected using methods disclosed in U.S. Pat. No. 6,384,301 where soybean meristem explants are wounded then inoculated and co-cultured with the soybean transformation vector, then transferred to selection media for 6-8 weeks to allow selection and growth of transgenic shoots.

Transgenic shoots producing roots are transferred to the greenhouse and potted in soil. Many transgenic events which survive to fertile transgenic plants that produce seeds and progeny plants do not exhibit an enhanced agronomic trait. The transgenic plants and seeds having the transgenic cells of this invention which have recombinant DNA imparting the enhanced agronomic traits are identified by screening for nitrogen use efficiency, yield, water use efficiency, cold tolerance and improved seed composition as reported in Example 7.

### Example 4

### Cotton Transgenic Plants with Enhanced Agronomic Traits

Cotton transformation is performed as generally described in WO0036911 and in U.S. Pat. No. 5,846,797. Transgenic cotton plants containing each of the recombinant DNA construct having a sequence of SEQ ID NO: 1 through SEQ ID NO: 95 are obtained by transforming with recombinant DNA from each of the genes identified in Table 1. Progeny transgenic plants are selected from a population of transgenic cotton events under specified growing conditions and are compared with control cotton plants. Control cotton plants are substantially the same cotton genotype but without the recombinant DNA, for example, either a parental cotton plant of the same genotype that was not transformed with the identical recombinant DNA or a negative isoline of the transformed plant. Additionally, a commercial cotton cultivar adapted to the geographical region and cultivation conditions, i.e. cotton variety ST474, cotton variety FM 958, and cotton variety Siokra L-23, are used to compare the relative performance of the transgenic cotton plants containing the recombinant DNA. The specified culture conditions are growing a first set of transgenic and control plants under “wet” conditions, i.e. irrigated in the range of 85 to 100 percent of evapotranspiration to provide leaf water potential of −14 to −18 bars, and growing a second set of transgenic and control plants under “dry” conditions; i.e: irrigated in the range of 40 to 60 percent of evapotranspiration to provide a leaf water potential of −21 to −25 bars. Pest control, such as weed and insect control is applied equally to both wet and dry treatments as needed. Data gathered during the trial includes weather records throughout the growing season including detailed records of rainfall; soil characterization information; any herbicide or insecticide applications; any gross agronomic differences observed such as leaf morphology, branching habit, leaf color, time to flowering, and fruiting pattern; plant height at various points during the trial; stand density; node and fruit number including node above white flower and node above crack boll measurements; and visual wilt scoring. Cotton boll samples are taken and analyzed for lint fraction and fiber quality. The cotton is harvested at the normal harvest timeframe for the trial area. Enhanced water use efficiency is indicated by increased yield, improved relative water content, enhanced leaf water potential; increased biomass, enhanced leaf extension rates, and improved fiber parameters.

The transgenic cotton plants of this invention are identified from among the transgenic cotton plants by agronomic trait screening as having increased yield and enhanced water use efficiency.

### Example 5

### Canola Transformation

This example illustrates plant transformation useful in producing the transgenic canola plants of this invention and the production and identification of transgenic seed for transgenic canola having enhanced water use efficiency, enhanced cold tolerance, increased yield, enhanced nitrogen use efficiency, enhanced seed protein and enhanced seed oil.

Tissues from in vitro grown canola seedlings are prepared and inoculated with overnight-grown Agrobacterium cells containing the recombinant DNA construct containing the DNA segment for the gene of interest cassette and a plant selectable marker cassette. Following co-cultivation with Agrobacterium, the infected tissues are allowed to grow on selection to promote growth of transgenic shoots, followed by growth of roots from the transgenic shoots. The selected planners are then transferred to the greenhouse and potted in soil. Molecular characterization are performed to confirm the presence of the gene of interest, and its expression in transgenic plants and progenies. Progeny transgenic plants are selected from a population of transgenic canola events under specified growing conditions and are compared with control canola plants: Control canola plants are substantially the same canola genotype but without the recombinant DNA, for example, either a parental canola plant of the same genotype that is not transformed with the identical recombinant DNA or a negative isoline of the transformed plant

Transgenic canola plant cells are transformed with recombinant DNA construct from each of the genes identified in Table 1. Transgenic progeny plants and seed of the transformed plant cells are screened for enhanced water use efficiency, enhanced cold tolerance, increased yield, enhanced nitrogen use efficiency, enhanced seed protein and enhanced seed oil as reported in Example 7.

### Example 6

### Homolog Identification

This example illustrates the identification of homologs of proteins encoded by the DNA identified in Table 1 which is used to provide transgenic seed and plants having enhanced agronomic traits. From the sequence of the homologs, homologous DNA sequence can be identified for preparing additional transgenic seeds and plants of this invention with enhanced agronomic traits.

An “All Protein Database” was constructed of known protein sequences using a proprietary sequence database and the National Center for Biotechnology Information (NCBI) non-redundant amino acid database (nr.aa). For each organism from which a polynucleotide sequence provided herein was obtained, an “Organism Protein Database” was constructed of known protein sequences of the organism; it is a subset of the All Protein Database based on the NCBI taxonomy ID for the organism.

The All Protein Database was queried using amino acid sequences provided herein as SEQ ID NO: 96 through SEQ ID NO: 193 using NCBI “blastp” program with E-value cutoff of 1 e-8. Up to 1000 top hits were kept, and separated by organism names. For each organism other than that of the query sequence, a list was kept for hits from the query organism itself with a more significant E-value than the best hit of the organism. The list contains likely duplicated genes of the polynucleotides provided herein, and is referred to as the Core List. Another list was kept for all the hits from each organism, sorted by E-value, and referred to as the Hit List.

The Organism Protein Database was queried using polypeptide sequences provided herein as SEQ ID NO: 96 through SEQ ID NO: 193 using NCBI “blastp” program with E-value cutoff of 1e-4. Up to 1000 top hits were kept. A BLAST searchable database was constructed based on these hits, and is referred to as “SubDB”, SubDB was queried with each sequence in the Hit List using NCBI “blastp” program with E-value cutoff of 1e-8. The hit with the best E-value was compared with the Core List from the corresponding organism. The hit is deemed a likely ortholog if it belongs to the Core List, otherwise it is deemed not a likely ortholog and there is no further search of sequences in the Hit List for the same organism. Homologs from a large number of distinct organisms were identified and are reported by amino acid sequences of SEQ ID NO: 194 through SEQ ID NO: 2166. The relationship of proteins of SEQ ID NO: 96 through 193 and homologs of SEQ ID NO: 194 through 2166 is identified in Table 8. The source organism for each homolog is found in the Sequence Listing.

TABLE 8
PEP SEQ ID NO: homolog SEQ ID Nos

| | | | | | | | | | | | | | |
|---|---|---|---|---|---|---|---|---|---|---|---|---|---|
| 96: | 1993 | 479 | 2151 | 697 | 1028 | 1041 | 1751 | 979 | 522 | 1247 | | | |
| 97: | 747 | 592 | 1014 | 1740 | 1904 | 1641 | 1354 | 1207 | 2134 | 1569 | 1635 | 407 | |
| | 602 | 635 | 397 | 2029 | 975 | | | | | | | | |
| 98: | 1246 | 1099 | 1100 | 2152 | 1152 | 1301 | 1474 | 1482 | 1994 | 1196 | 737 | 736 | |
| | 324 | 1078 | 1076 | 615 | 339 | 322 | 335 | 775 | 608 | 611 | 613 | 1745 | |
| | 1744 | 1742 | 1697 | 486 | 2049 | 1971 | 454 | 1472 | 789 | 1829 | 1058 | 1941 | |
| | 368 | 574 | 1555 | 1416 | 1228 | 1020 | 456 | 205 | 1081 | 2047 | 1746 | 489 | |
| | 821 | 1409 | 2081 | 534 | 330 | 1884 | 2138 | 1748 | 1621 | 1361 | 1505 | 1199 | |
| | 1985 | 1756 | 371 | 693 | 1702 | 1287 | 1766 | 1571 | 1573 | 550 | 552 | 1726 | |
| | 1891 | 1727 | 1633 | 634 | 253 | 457 | 288 | 290 | 398 | 405 | 401 | 1516 | |
| | 1356 | 2038 | 551 | 417 | 1553 | 2055 | 863 | 1284 | 1282 | 1917 | 1102 | 735 | |
| | 768 | 531 | 720 | 616 | 466 | 772 | 1708 | | | | | | |
| 99: | 1041 | 1906 | 491 | 927 | 1788 | 1116 | 404 | 1636 | 1231 | 1676 | 1684 | 139 | |
| | 136 | 135 | 133 | 1132 | 1026 | | | | | | | | |
| 100: | 1041 | 1906 | 491 | 927 | 1788 | 1116 | 404 | 1636 | 1231 | 1676 | 1684 | 139 | |
| | 136 | 135 | 133 | 1132 | 1026 | | | | | | | | |
| 101: | 894 | 956 | 458 | 1085 | 809 | 1448 | 102 | 1855 | 797 | 1620 | 714 | 505 | |
| | 1339 | 927 | 715 | 979 | 1805 | 639 | 384 | 578 | | | | | |
| 102: | 956 | 1085 | 458 | 809 | 1145 | 1448 | 505 | 927 | 715 | 570 | 459 | 1018 | |
| | 1104 | 477 | 810 | 455 | 654 | 101 | 578 | 1704 | | | | | |
| 103: | 1041 | 1906 | 491 | 927 | 1788 | 1116 | 404 | 1636 | 1231 | 1676 | 1684 | 139 | |
| | 136 | 135 | 133 | 1132 | 1026 | | | | | | | | |
| 104: | 781 | 1325 | 2061 | 1348 | 1350 | 1346 | 467 | 256 | 903 | 915 | 1759 | 1882 | |
| | 1432 | 1433 | 734 | 669 | 1639 | 575 | 1203 | 544 | 525 | 1547 | 970 | 1403 | |
| | 1665 | 1366 | 652 | 1566 | 920 | 1984 | 470 | 319 | 1288 | 1290 | 1375 | 1296 | |
| | 1395 | 1328 | 1330 | 1311 | 292 | 661 | 2131 | 2146 | 2085 | 542 | 916 | 1519 | |
| | 1367 | 1364 | 1313 | 1314 | 1324 | 1355 | 1352 | 1293 | 1294 | 1378 | 1401 | 1399 | |
| | 1310 | 2068 | 2082 | 1160 | 1773 | | | | | | | | |
| 105: | 1497 | 1872 | 730 | 361 | 732 | 2066 | 1954 | 690 | 201 | 1359 | 473 | 1879 | |
| | 745 | 1129 | 411 | 415 | 1715 | 2144 | 1074 | 1476 | 1417 | | | | |
| 106: | 806 | 869 | 1412 | 264 | 265 | 248 | 246 | 1096 | 286 | 312 | 238 | 1864 | |
| | 1860 | 1243 | 1261 | 744 | 428 | 930 | 496 | 629 | 851 | 852 | 2148 | 497 | |
| | 516 | 933 | 1814 | 1478 | 1463 | 573 | 377 | 641 | 867 | 665 | 221 | 1455 | |
| | 991 | 653 | 601 | 1524 | 1418 | 526 | 883 | 2110 | 1185 | 1368 | 1630 | 1441 | |
| | 2004 | 1771 | 919 | 1098 | 1138 | 424 | 1731 | 1379 | 413 | 1186 | 778 | 833 | |
| | 1172 | 2034 | 1607 | 1791 | 147 | 144 | 143 | 887 | 504 | 988 | 228 | 936 | |
| | 499 | 1931 | 403 | 382 | 609 | 680 | 733 | 982 | 1725 | 1713 | 1711 | 1710 | |
| | 414 | 1044 | | | | | | | | | | | |
| 107: | 1369 | 1400 | 831 | 344 | 218 | 588 | 1863 | 1489 | 2031 | 1450 | 375 | 1709 | |
| | 798 | 1837 | 1681 | 543 | 1500 | 1005 | 1159 | 1900 | | | | | |
| 108: | 1936 | 853 | 1935 | 2105 | 350 | 594 | 893 | 2101 | 1268 | 1233 | 866 | 1422 | |
| 109: | 1151 | 1167 | 1166 | 1178 | 1358 | 630 | 500 | 538 | 535 | 1413 | 1564 | 1721 | |
| | 198 | 1898 | 723 | 1411 | 1019 | 1316 | 1331 | 1318 | 1999 | 2007 | 1822 | 519 | |
| | 709 | 329 | 840 | 1511 | 1502 | 766 | 671 | 1951 | 1436 | 501 | 610 | 612 | |
| | 539 | 1414 | 648 | 1263 | 997 | | | | | | | | |
| 110: | 769 | 1861 | 1156 | 1828 | 1093 | 568 | 677 | 1659 | 252 | 250 | 370 | 276 | |
| | 197 | 1133 | 275 | 262 | 261 | 259 | 257 | 241 | 239 | 236 | 889 | 1012 | |
| | 1209 | | | | | | | | | | | | |
| 111: | 781 | 1325 | 2061 | 1348 | 1350 | 1346 | 1332 | 196 | 903 | 915 | 1759 | 1882 | |
| | 1639 | 2006 | 667 | 2035 | 652 | 1566 | 1169 | 830 | 1220 | 2051 | 920 | 470 | |
| | 1105 | 1103 | 1290 | 1288 | 1375 | 1370 | 1296 | 1395 | 1328 | 1330 | 1311 | 509 | |
| | 579 | 1488 | 628 | 292 | 1875 | 952 | 1504 | 1803 | 779 | 1430 | 285 | 1857 | |
| | 1598 | 1718 | 796 | 1101 | 1739 | 423 | 542 | 916 | 1519 | 945 | 1313 | 1364 | |
| | 1367 | 1324 | 1314 | 1352 | 1355 | 1293 | 1294 | 1378 | 1401 | 1399 | 1310 | 2082 | |
| | 2068 | 1597 | 1160 | 1773 | 1599 | 1586 | 1582 | 1976 | 1580 | 1722 | 1545 | | |
| 112: | 823 | 1253 | 1750 | 247 | 1859 | 1753 | 2147 | 1752 | 1544 | 688 | 1631 | 1347 | |
| | 1769 | 875 | 510 | 503 | 1663 | 374 | 722 | 752 | 942 | 2128 | 512 | 987 | |
| | 1298 | 1066 | | | | | | | | | | | |
| 113: | 1396 | 1221 | 433 | 326 | 406 | 2093 | 1273 | 1670 | 1655 | 1672 | 2031 | 1147 | |
| | 338 | 1712 | 553 | 619 | 986 | 1479 | 792 | 1837 | 1681 | 543 | 741 | | |
| 114: | 1144 | 1143 | 1813 | 386 | 995 | 502 | 708 | 707 | 1886 | 206 | 1007 | 1279 | |
| | 1809 | 1389 | 1657 | 1644 | 882 | 1075 | 1069 | 911 | 1867 | 1023 | 223 | 1934 | |
| | 1541 | 356 | 1960 | 2062 | 914 | 1051 | 2057 | 672 | 476 | 394 | 438 | 1637 | |
| | 605 | 1079 | 1513 | 1795 | 1862 | 2028 | 1970 | 1983 | 646 | 304 | 971 | 1577 | |
| | 1539 | 755 | 1624 | 1895 | 2098 | 913 | | | | | | | |
| 115: | 800 | 2016 | 213 | 1056 | 254 | 799 | 1217 | 600 | 334 | 848 | 1134 | 444 | |
| | 185 | 1950 | 2046 | 1468 | 1888 | 1124 | 1923 | 953 | 985 | 1587 | 1000 | 999 | |
| | 1001 | 716 | 1877 | 828 | 1887 | 1937 | 621 | | | | | | |
| 116: | 800 | 2016 | 213 | 1056 | 799 | 1217 | 848 | 600 | 334 | 1134 | 444 | 1139 | |
| | 185 | 1950 | 904 | 985 | 1587 | 1000 | 999 | 372 | 1162 | 1887 | 1937 | 1768 | |
| | 1091 | | | | | | | | | | | | |
| 117: | 203 | 711 | 1833 | 1765 | 1956 | 1295 | 410 | 1050 | 822 | 1496 | 1781 | 762 | |
| | 1095 | 981 | 1006 | 1097 | 1827 | 1611 | 1632 | 559 | 527 | 1512 | 445 | 1473 | |
| 118: | 853 | 1935 | 2105 | 393 | 1499 | 993 | 1595 | 1514 | 478 | 299 | 1873 | 1148 | |
| | 893 | 1552 | 803 | 2090 | 1646 | 1533 | 1615 | 1260 | 866 | | | | |
| 119: | 1517 | 1030 | 1583 | 359 | 655 | 1521 | 327 | 1276 | 815 | 1323 | 245 | 318 | |
| | 962 | 2078 | 549 | 888 | 296 | 2084 | 513 | 422 | 378 | 1398 | 1501 | 1785 | |
| | 679 | 2037 | 864 | 1986 | 390 | 463 | 1525 | 1576 | 306 | 1674 | 1245 | 380 | |
| | 1142 | 367 | 1821 | 1437 | 767 | 923 | 1878 | 2043 | 1381 | 1678 | 465 | 1550 | |
| | 1618 | 902 | 137 | | | | | | | | | | |
| 120: | 150 | 121 | 1491 | 1380 | | | | | | | | | |
| 121: | 120 | 150 | 1491 | 1380 | | | | | | | | | |
| 122: | 659 | 1724 | 1835 | 507 | 1242 | 836 | 738 | 895 | 1572 | 1625 | 1218 | 879 | |
| | 1990 | 365 | 1299 | 1425 | 597 | 790 | 1338 | 1945 | 1691 | 1737 | 2129 | 1690 | |
| | 924 | 791 | 855 | 123 | 2154 | 1852 | 837 | 1981 | 2112 | | | | |
| 123: | 659 | 1724 | 1835 | 507 | 1242 | 836 | 738 | 895 | 1572 | 1625 | 1218 | 879 | |
| | 1990 | 365 | 1299 | 1425 | 597 | 790 | 1338 | 1945 | 1691 | 1737 | 2129 | 1690 | |
| | 924 | 791 | 855 | 122 | 211 | 2154 | 1852 | 837 | 1981 | 2112 | | | |
| 124: | 1177 | 1754 | 521 | 1254 | 2067 | 471 | 2012 | 1673 | 834 | 1280 | 1464 | 400 | |
| | 807 | 622 | 1027 | 1939 | 1556 | 426 | 565 | 1360 | 1452 | 493 | 1569 | 1200 | |
| | 1892 | 321 | 2071 | 427 | 1831 | 1557 | 1701 | 1890 | 1889 | 785 | 1334 | 1149 | |
| | 1255 | 1438 | 1554 | 1838 | | | | | | | | | |
| 125: | 1475 | 1302 | 388 | 2165 | 2070 | 2073 | 2065 | 865 | 1077 | 484 | 589 | 487 | |
| | 604 | 1067 | 1068 | 760 | 994 | 585 | 495 | 890 | 1885 | 876 | 1285 | 2033 | |
| | 222 | 1762 | 1015 | 801 | 839 | 842 | 1871 | 844 | 435 | 857 | 859 | 650 | |
| | 1237 | 1238 | 861 | 1111 | 2111 | 432 | 694 | 1929 | 314 | 1170 | 1942 | 1908 | |
| | 1560 | 1216 | 1964 | 537 | 311 | 825 | 841 | 1530 | 1002 | 1909 | 443 | 1682 | |
| | 664 | 572 | 2083 | 1070 | 1153 | | | | | | | | |
| 126: | 894 | 956 | 2013 | 430 | 1041 | 1448 | 713 | 1021 | 591 | 325 | 786 | 928 | |
| | 1034 | 1522 | 1506 | 1743 | 1905 | 522 | | | | | | | |
| 127: | 1503 | 1108 | 1543 | 1315 | 1881 | 1922 | 1239 | 2030 | 266 | 873 | | | |
| 128: | 1528 | 387 | 206 | 1007 | 271 | 607 | 1692 | 1991 | 1848 | 1345 | 963 | 1651 | |
| | 1213 | 249 | 1903 | 402 | 186 | 153 | 773 | 1901 | 1874 | 412 | 1190 | 480 | |
| | 1297 | 1003 | 1122 | 898 | 1540 | 660 | 564 | 1616 | 1051 | 555 | 683 | 1987 | |
| | 273 | 244 | 672 | 438 | 1384 | 1520 | 698 | 793 | 2052 | 1173 | 756 | 336 | |
| | 1269 | 1094 | 2145 | 1234 | 1807 | 943 | 1949 | 776 | 704 | 1486 | 1961 | 1776 | |
| | 482 | 625 | 1775 | 483 | 1526 | | | | | | | | |
| 129: | 1528 | 387 | 206 | 1007 | 271 | 607 | 1692 | 1991 | 1848 | 1345 | 963 | 1651 | |
| | 1213 | 249 | 1903 | 402 | 186 | 153 | 773 | 1901 | 1874 | 412 | 1190 | 480 | |
| | 1297 | 1003 | 1122 | 898 | 1540 | 660 | 564 | 1616 | 1051 | 555 | 683 | 1987 | |
| | 273 | 244 | 672 | 438 | 1384 | 1520 | 698 | 793 | 2052 | 1173 | 756 | 336 | |
| | 1269 | 1094 | 2145 | 1234 | 1807 | 943 | 1949 | 776 | 704 | 1486 | 1961 | 1776 | |
| | 482 | 625 | 1775 | 483 | 1526 | | | | | | | | |
| 130: | 1528 | 387 | 206 | 1007 | 271 | 607 | 1692 | 1991 | 1848 | 1345 | 963 | 1651 | |
| | 1213 | 249 | 1903 | 402 | 186 | 153 | 773 | 1901 | 1874 | 412 | 1190 | 480 | |
| | 1297 | 1003 | 1122 | 898 | 1540 | 660 | 564 | 1616 | 1051 | 555 | 683 | 1987 | |
| | 273 | 244 | 672 | 438 | 1384 | 1520 | 698 | 793 | 2052 | 1173 | 756 | 336 | |
| | 1269 | 1094 | 2145 | 1234 | 1807 | 943 | 1949 | 776 | 704 | 1486 | 1961 | 1776 | |
| | 482 | 625 | 1775 | 483 | 1526 | | | | | | | | |
| 131: | 1528 | 387 | 206 | 1007 | 271 | 607 | 1692 | 1991 | 1848 | 1345 | 963 | 1651 | |
| | 1213 | 249 | 1903 | 402 | 186 | 153 | 773 | 1901 | 1874 | 412 | 1190 | 480 | |
| | 1297 | 1003 | 1122 | 898 | 1540 | 660 | 564 | 1616 | 1051 | 555 | 683 | 1987 | |
| | 273 | 244 | 672 | 438 | 1384 | 1520 | 698 | 793 | 2052 | 1173 | 756 | 336 | |
| | 1269 | 1094 | 2145 | 1234 | 1807 | 943 | 1949 | 776 | 704 | 1486 | 1961 | 1776 | |
| | 482 | 625 | 1775 | 483 | 1526 | | | | | | | | |
| 132: | 1528 | 387 | 206 | 1007 | 271 | 607 | 1692 | 1991 | 1848 | 1345 | 963 | 1651 | |
| | 1213 | 249 | 1903 | 402 | 186 | 153 | 773 | 1901 | 1874 | 412 | 1190 | 480 | |
| | 1297 | 1003 | 1122 | 898 | 1540 | 660 | 564 | 1616 | 1051 | 555 | 683 | 1987 | |
| | 273 | 244 | 672 | 438 | 1384 | 1520 | 698 | 793 | 2052 | 1173 | 756 | 336 | |
| | 1269 | 1094 | 2145 | 1234 | 1807 | 943 | 1949 | 776 | 704 | 1486 | 1961 | 1776 | |
| | 482 | 625 | 1775 | 483 | 1526 | | | | | | | | |
| 133: | 956 | 1906 | 927 | 979 | 1788 | 1116 | 404 | 1636 | 1231 | 1026 | 103 | 100 | |
| | 99 | 1834 | | | | | | | | | | | |
| 134: | 1157 | 758 | 451 | 548 | 1281 | 832 | 1136 | 1816 | 1804 | 710 | 858 | 289 | |
| | 618 | 1049 | 1933 | 532 | 1567 | 1256 | 878 | 620 | 1031 | 2118 | | | |
| 135: | 956 | 1906 | 927 | 979 | 1788 | 1116 | 404 | 1636 | 1231 | 1026 | 103 | 100 | |
| | 99 | 1834 | | | | | | | | | | | |
| 136: | 956 | 1906 | 927 | 979 | 1788 | 1116 | 404 | 1636 | 1231 | 1026 | 103 | 100 | |
| | 99 | 1834 | | | | | | | | | | | |
| 137: | 1517 | 1030 | 1583 | 359 | 655 | 1521 | 327 | 1276 | 815 | 1323 | 245 | 318 | |
| | 962 | 2078 | 549 | 888 | 296 | 2084 | 513 | 422 | 378 | 1398 | 1501 | 1785 | |
| | 679 | 2037 | 864 | 1986 | 390 | 463 | 1525 | 1576 | 306 | 1674 | 1245 | 380 | |
| | 1142 | 367 | 1821 | 1437 | 767 | 923 | 1878 | 2043 | 1381 | 1678 | 465 | 1550 | |
| | 1618 | 902 | 119 | | | | | | | | | | |
| 138: | 1144 | 708 | 202 | 364 | 1321 | 219 | 216 | 1541 | 356 | 603 | 488 | 587 | |
| | 581 | 2126 | 394 | 438 | 1272 | 1666 | 225 | 651 | 1604 | 1602 | 989 | 1060 | |
| | 1594 | 243 | 1082 | 1054 | 1970 | 1865 | | | | | | | |
| 139: | 956 | 1906 | 927 | 979 | 1788 | 1116 | 404 | 1636 | 1231 | 1026 | 103 | 100 | |
| | 99 | 1834 | | | | | | | | | | | |
| 140: | 416 | 1799 | 662 | 298 | 1278 | 1126 | 1694 | 1176 | 1187 | 1819 | 2119 | 1397 | |
| | 468 | 1664 | 1493 | 932 | 1393 | 1498 | 2088 | 1811 | 1259 | 1184 | 369 | 1008 | |
| | 1009 | 1966 | 395 | 1036 | 1037 | 1039 | 1033 | 1995 | 1992 | 1040 | 1071 | 1065 | |
| | 1155 | 1141 | 352 | 354 | 1926 | 1826 | 1661 | 1053 | 301 | 1642 | 233 | 1627 | |
| | 2048 | 1593 | 1613 | 1483 | 1667 | 1010 | 437 | 1125 | 1080 | 1092 | 529 | 331 | |
| | 347 | 1982 | 1165 | 1568 | 1542 | 1451 | | | | | | | |
| 141: | 1320 | 2158 | 541 | 295 | 1150 | 984 | 284 | 743 | 1714 | 1728 | 2008 | 1136 | |
| | 1823 | 847 | 1508 | 2115 | 2130 | 1121 | 1977 | 1802 | 481 | 1466 | 1244 | 1140 | |
| | 717 | 1996 | 1979 | 461 | | | | | | | | | |
| 142: | 806 | 1210 | 871 | 1412 | 248 | 265 | 264 | 246 | 2075 | 286 | 312 | 238 | |
| | 1243 | 744 | 428 | 629 | 1131 | 2148 | 1110 | 1426 | 497 | 933 | 1814 | 1927 | |
| | 1924 | 641 | 1918 | 1578 | 926 | 958 | 1847 | 1197 | 2096 | 939 | 1368 | 1630 | |
| | 1441 | 886 | 490 | 1731 | 1729 | 782 | 557 | 1227 | 1419 | 1179 | 2045 | 1792 | |
| | 1774 | 1186 | 1219 | 1423 | 977 | 1607 | 1791 | 1763 | 1761 | 1789 | 1777 | 682 | |
| | 644 | 268 | 226 | 623 | 829 | 1793 | 1836 | 1265 | 1790 | 263 | 258 | 260 | |
| | 731 | 566 | 1376 | 1720 | 447 | 1579 | 850 | 1953 | 676 | 849 | 1308 | 1869 | |
| | 2092 | 567 | 967 | 2009 | 499 | 936 | 1931 | 403 | 382 | 609 | 680 | 733 | |
| | 982 | 1725 | 1713 | 1711 | 1710 | 1693 | 1214 | | | | | | |
| 143: | 806 | 1191 | 1194 | 869 | 868 | 1412 | 1303 | 264 | 265 | 248 | 246 | 1096 | |
| | 286 | 312 | 315 | 238 | 1864 | 1860 | 1243 | 1261 | 744 | 428 | 930 | 496 | |
| | 629 | 851 | 852 | 1127 | 1128 | 2148 | 497 | 516 | 933 | 1017 | 1814 | 1478 | |
| | 1463 | 1212 | 573 | 377 | 641 | 653 | 601 | 867 | 991 | 1455 | 665 | 1524 | |
| | 221 | 1418 | 526 | 883 | 2110 | 1185 | 1630 | 1368 | 1441 | 2004 | 1771 | 919 | |
| | 1098 | 316 | 881 | 1138 | 424 | 816 | 1731 | 1792 | 1774 | 1186 | 464 | 778 | |
| | 1916 | 1955 | 1219 | 827 | 826 | 724 | 1423 | 725 | 727 | 833 | 1172 | 2034 | |
| | 977 | 1607 | 1791 | 228 | 106 | 1608 | 1044 | 1154 | 936 | 499 | 1931 | 403 | |
| | 382 | 609 | 680 | 733 | 982 | 1725 | 1713 | 1711 | 1710 | 414 | 887 | | |
| 144: | 806 | 1191 | 1194 | 869 | 868 | 1412 | 1303 | 264 | 265 | 248 | 246 | 1096 | |
| | 286 | 312 | 315 | 238 | 1864 | 1860 | 1243 | 1261 | 744 | 428 | 930 | 496 | |
| | 629 | 851 | 852 | 1127 | 1128 | 2148 | 497 | 516 | 933 | 1017 | 1814 | 1478 | |
| | 1463 | 1212 | 573 | 377 | 641 | 653 | 601 | 867 | 991 | 1455 | 665 | 1524 | |
| | 221 | 1418 | 526 | 883 | 2110 | 1185 | 1630 | 1368 | 1441 | 2004 | 1771 | 919 | |
| | 1098 | 316 | 881 | 1138 | 424 | 816 | 1731 | 1792 | 1774 | 1186 | 464 | 778 | |
| | 1916 | 1955 | 1219 | 827 | 826 | 724 | 1423 | 725 | 727 | 833 | 1172 | 2034 | |
| | 977 | 1607 | 1791 | 228 | 106 | 1608 | 1044 | 1154 | 936 | 499 | 1931 | 403 | |
| | 382 | 609 | 680 | 733 | 982 | 1725 | 1713 | 1711 | 1710 | 414 | 887 | | |
| 145: | 1570 | 294 | 1640 | 2053 | 1248 | 1703 | 1252 | 547 | 1201 | 1362 | 1386 | 2132 | |
| | 712 | 1327 | 453 | 363 | 1841 | 379 | 212 | 921 | 2060 | 1340 | 446 | 998 | |
| | 891 | 961 | 627 | 1928 | 910 | 805 | 1064 | 1899 | 1469 | 2108 | 1716 | 237 | |
| | 1972 | 1038 | 949 | 1732 | 1289 | 1808 | 1824 | 2010 | 695 | 560 | 1029 | 1946 | |
| | 1089 | 657 | 1088 | 158 | 1730 | 328 | | | | | | | |
| 146: | 769 | 281 | 279 | 282 | 267 | 1861 | 2102 | 978 | 1106 | 569 | 421 | 1638 | |
| | 360 | 2143 | 370 | 2032 | 1283 | 276 | 272 | 270 | 261 | 241 | 275 | 262 | |
| | 257 | 236 | 259 | 239 | 889 | 1012 | | | | | | | |
| 147: | 806 | 1191 | 1194 | 869 | 868 | 1412 | 1303 | 264 | 265 | 248 | 246 | 1096 | |
| | 286 | 312 | 315 | 238 | 1864 | 1860 | 1243 | 1261 | 744 | 428 | 930 | 496 | |
| | 629 | 851 | 852 | 1127 | 1128 | 2148 | 497 | 516 | 933 | 1017 | 1814 | 1478 | |
| | 1463 | 1212 | 573 | 377 | 641 | 653 | 601 | 867 | 991 | 1455 | 665 | 1524 | |
| | 221 | 1418 | 526 | 883 | 2110 | 1185 | 1630 | 1368 | 1441 | 2004 | 1771 | 919 | |
| | 1098 | 316 | 881 | 1138 | 424 | 816 | 1731 | 1792 | 1774 | 1186 | 464 | 778 | |
| | 1916 | 1955 | 1219 | 827 | 826 | 724 | 1423 | 725 | 727 | 833 | 1172 | 2034 | |
| | 977 | 1607 | 1791 | 228 | 106 | 1608 | 1044 | 1154 | 936 | 499 | 1931 | 403 | |
| | 382 | 609 | 680 | 733 | 982 | 1725 | 1713 | 1711 | 1710 | 414 | 887 | | |
| 148: | 210 | 1183 | 1548 | 1291 | 1471 | 2140 | 436 | 1120 | 1119 | 1115 | 1118 | 2141 | |
| | 687 | 1656 | 1534 | 1286 | 905 | 1251 | 1372 | 313 | 1603 | 1300 | 638 | 1181 | |
| | 1198 | 220 | | | | | | | | | | | |
| 149: | 1277 | 524 | 955 | 658 | 1609 | 317 | 1180 | 1536 | 355 | 353 | 357 | 2005 | |
| | 399 | 229 | 645 | 425 | 1561 | 1958 | 1894 | 1312 | 1940 | 1783 | 1250 | 1390 | |
| | 1391 | 1507 | 917 | 227 | 640 | 996 | 2106 | 1446 | 518 | 1649 | 606 | 794 | |
| | 520 | 1610 | 1492 | 1208 | 1223 | 1800 | 1851 | 1706 | 1914 | 2039 | 941 | 1870 | |
| | 1698 | 434 | 2040 | 293 | 1778 | 1733 | 1322 | 1059 | 449 | 376 | 1846 | 2103 | |
| | 1025 | 1107 | | | | | | | | | | | |
| 150: | 120 | 121 | 1491 | 1380 | | | | | | | | | |
| 151: | 696 | 333 | 1013 | 1336 | 1257 | 1606 | 804 | 392 | 506 | 835 | 1193 | 593 | |
| | 1114 | 637 | 974 | 556 | 1734 | 906 | 701 | 1371 | 1086 | 1683 | 2095 | 1612 | |
| | 862 | 1896 | 362 | 419 | 1974 | 813 | 765 | | | | | | |
| 152: | 1369 | 1400 | 831 | 344 | 218 | 588 | 1863 | 1489 | 2031 | 1450 | 375 | 1709 | |
| | 798 | 1837 | 1681 | 543 | 1500 | 1005 | 1159 | 1900 | | | | | |
| 153: | 1144 | 1143 | 1813 | 386 | 995 | 502 | 1668 | 1007 | 1279 | 271 | 1651 | 1024 | |
| | 1541 | 356 | 1540 | 660 | 1689 | 1051 | 488 | 2121 | 555 | 683 | 1987 | 581 | |
| | 132 | 131 | 130 | 129 | 128 | 973 | 672 | 438 | 1272 | 1666 | 1384 | 1520 | |
| | 698 | 1173 | 1513 | 336 | 1269 | 756 | 943 | 1949 | 776 | 243 | 1241 | 1970 | |
| | 646 | 304 | 402 | | | | | | | | | | |
| 154: | 1405 | 1794 | 1087 | 2097 | 1304 | 1035 | 681 | 969 | 1628 | 2163 | 2100 | 452 | |
| | 1055 | 636 | 899 | 1083 | 885 | 2077 | 1549 | 582 | 1700 | 1947 | 1932 | 1084 | |
| 155: | 781 | 1325 | 2061 | 1348 | 1350 | 1346 | 467 | 256 | 903 | 915 | 1432 | 1433 | |
| | 1685 | 1688 | 1699 | 1669 | 1806 | 159 | 111 | 1224 | 1722 | 1645 | 748 | 1545 | |
| | 1853 | 1707 | 345 | 1798 | 429 | 787 | 278 | 1639 | 901 | 2006 | 614 | 1236 | |
| | 544 | 525 | 1547 | 667 | 1566 | 1169 | 1220 | 830 | 2051 | 920 | 1984 | 2036 | |
| | 470 | 319 | 1105 | 1288 | 1290 | 1375 | 1296 | 1395 | 1328 | 1330 | 1311 | 579 | |
| | 292 | 1875 | 1718 | 796 | 542 | 916 | 1519 | 945 | 1313 | 1367 | 1364 | 1314 | |
| | 1324 | 1352 | 1355 | 1293 | 1294 | 1378 | 1401 | 1399 | 1310 | 2068 | 2082 | 1597 | |
| | 1160 | 1773 | 1586 | 1599 | 1976 | 1582 | 1580 | 1817 | 673 | | | | |
| 156: | 586 | 1434 | 2042 | 280 | 1523 | 2079 | 1647 | 705 | 817 | 1820 | 1915 | 617 | |
| | 1830 | 1135 | 2125 | 2149 | 1858 | 492 | 1757 | 819 | 918 | 818 | 759 | 692 | |
| 157: | 1517 | 1030 | 1583 | 359 | 655 | 1705 | 740 | 439 | 2020 | 1973 | 908 | 1090 | |
| | 1796 | 1696 | 962 | 318 | 2084 | 2017 | 1057 | 1117 | 422 | 378 | 684 | 554 | |
| | 1501 | 474 | 679 | 1910 | 864 | 880 | 624 | 1584 | 1307 | 234 | 1204 | 306 | |
| | 546 | 1674 | 1782 | 1245 | 396 | 1189 | 1211 | 678 | 367 | 1779 | 1821 | 1437 | |
| | 870 | 1878 | 2043 | 2058 | 1686 | 445 | 1381 | 1678 | 558 | 465 | 1550 | 1618 | |
| | 892 | 1680 | | | | | | | | | | | |
| 158: | 1570 | 294 | 1640 | 2053 | 1248 | 1252 | 1703 | 547 | 1201 | 1362 | 1386 | 2132 | |
| | 712 | 1327 | 453 | 363 | 212 | 1841 | 379 | 2060 | 446 | 998 | 1340 | 961 | |
| | 627 | 910 | 805 | 1928 | 1899 | 1469 | 1052 | 2108 | 1716 | 237 | 1972 | 1038 | |
| | 949 | 1289 | 1808 | 1824 | 2010 | 695 | 560 | 1029 | 1946 | 1089 | 657 | 1088 | |
| | 1730 | 328 | | | | | | | | | | | |
| 159: | 781 | 1325 | 2061 | 1348 | 1350 | 1346 | 1332 | 196 | 903 | 915 | 1759 | 1882 | |
| | 1639 | 2006 | 667 | 2035 | 652 | 1566 | 1169 | 830 | 1220 | 2051 | 920 | 470 | |
| | 1105 | 1103 | 1290 | 1288 | 1375 | 1370 | 1296 | 1395 | 1328 | 1330 | 1311 | 509 | |
| | 579 | 1488 | 628 | 292 | 1875 | 952 | 1504 | 1803 | 779 | 1430 | 285 | 1857 | |
| | 1598 | 1718 | 796 | 1101 | 1739 | 423 | 542 | 916 | 1519 | 945 | 1313 | 1364 | |
| | 1367 | 1324 | 1314 | 1352 | 1355 | 1293 | 1294 | 1378 | 1401 | 1399 | 1310 | 2082 | |
| | 2068 | 1597 | 1160 | 1773 | 1599 | 1586 | 1582 | 1976 | 1580 | 1722 | 1545 | | |
| 160: | 1570 | 294 | 1640 | 2053 | 1248 | 1252 | 1703 | 547 | 1201 | 1362 | 1386 | 2132 | |
| | 712 | 1327 | 453 | 363 | 1559 | 2091 | 1735 | 1596 | 1402 | 385 | 1842 | 1695 | |
| | 703 | 1484 | 1373 | 961 | 1658 | 563 | 2104 | 702 | 1736 | 1662 | 1899 | 1469 | |
| | 1052 | 1767 | 699 | 1801 | 237 | 1716 | 240 | 242 | 1972 | 1038 | 528 | 2021 | |
| | 1249 | 300 | 1824 | 2010 | 695 | 560 | 1029 | 1946 | 1089 | 657 | 1088 | 1854 | |
| | 164 | 162 | 161 | 165 | 420 | 1113 | 1738 | 685 | 1843 | 1415 | | | |
| 161: | 1570 | 294 | 1640 | 2053 | 1248 | 1252 | 1703 | 547 | 1201 | 1362 | 1386 | 2132 | |
| | 712 | 1327 | 453 | 363 | 1559 | 2091 | 1735 | 1596 | 1402 | 385 | 1842 | 1695 | |
| | 703 | 1484 | 1373 | 961 | 1658 | 563 | 2104 | 702 | 1736 | 1662 | 1899 | 1469 | |
| | 1052 | 1767 | 699 | 1801 | 237 | 1716 | 240 | 242 | 1972 | 1038 | 528 | 2021 | |
| | 1249 | 300 | 1824 | 2010 | 695 | 560 | 1029 | 1946 | 1089 | 657 | 1088 | 165 | |
| | 164 | 162 | 1854 | 160 | 420 | 1113 | 1738 | 685 | 1843 | 1415 | | | |
| 162: | 1570 | 294 | 1640 | 2053 | 1248 | 1252 | 1703 | 547 | 1201 | 1362 | 1386 | 2132 | |
| | 712 | 1327 | 453 | 363 | 1559 | 2091 | 1735 | 1596 | 1402 | 385 | 1842 | 1695 | |
| | 703 | 1484 | 1373 | 961 | 1658 | 563 | 2104 | 702 | 1736 | 1662 | 1899 | 1469 | |
| | 1052 | 1767 | 699 | 1801 | 237 | 1716 | 240 | 242 | 1972 | 1038 | 528 | 2021 | |
| | 1249 | 300 | 1824 | 2010 | 695 | 560 | 1029 | 1946 | 1089 | 657 | 1088 | 165 | |
| | 164 | 161 | 1854 | 160 | 420 | 1113 | 1738 | 685 | 1843 | 1415 | | | |
| 163: | 1605 | | | | | | | | | | | | |
| 164: | 1570 | 294 | 1640 | 2053 | 1248 | 1252 | 1703 | 547 | 1201 | 1362 | 1386 | 2132 | |
| | 712 | 1327 | 453 | 363 | 1559 | 2091 | 1735 | 1596 | 1402 | 385 | 1842 | 1695 | |
| | 703 | 1484 | 1373 | 961 | 1658 | 563 | 2104 | 702 | 1736 | 1662 | 1899 | 1469 | |
| | 1052 | 1767 | 699 | 1801 | 237 | 1716 | 240 | 242 | 1972 | 1038 | 528 | 2021 | |
| | 1249 | 300 | 1824 | 2010 | 695 | 560 | 1029 | 1946 | 1089 | 657 | 1088 | 162 | |
| | 165 | 161 | 1854 | 160 | 420 | 1113 | 1738 | 685 | 1843 | 1415 | | | |
| 165: | 1570 | 294 | 1640 | 2053 | 1248 | 1252 | 1703 | 547 | 1201 | 1362 | 1386 | 2132 | |
| | 712 | 1327 | 453 | 363 | 1559 | 2091 | 1735 | 1596 | 1402 | 385 | 1842 | 1695 | |
| | 703 | 1484 | 1373 | 961 | 1658 | 563 | 2104 | 702 | 1736 | 1662 | 1899 | 1469 | |
| | 1052 | 1767 | 699 | 1801 | 237 | 1716 | 240 | 242 | 1972 | 1038 | 528 | 2021 | |
| | 1249 | 300 | 1824 | 2010 | 695 | 560 | 1029 | 1946 | 1089 | 657 | 1088 | 161 | |
| | 162 | 164 | 1854 | 160 | 420 | 1113 | 1738 | 685 | 1843 | 1415 | | | |
| 166: | 1144 | 708 | 202 | 364 | 1321 | 219 | 216 | 1541 | 356 | 603 | 488 | 587 | |
| | 581 | 2126 | 394 | 438 | 1272 | 1666 | 225 | 651 | 1604 | 1602 | 989 | 1060 | |
| | 1594 | 243 | 1082 | 1054 | 1970 | 1865 | | | | | | | |
| 167: | 1177 | 1754 | 521 | 1254 | 2067 | 471 | 2012 | 1673 | 834 | 1280 | 1464 | 400 | |
| | 807 | 622 | 1027 | 1939 | 1556 | 426 | 565 | 1360 | 1452 | 493 | 1569 | 1200 | |
| | 1892 | 321 | 2071 | 427 | 1831 | 1557 | 1701 | 1890 | 1889 | 785 | 1334 | 1149 | |
| | 1255 | 1438 | 1554 | 1838 | | | | | | | | | |
| 168: | 1475 | 1302 | 388 | 2165 | 2070 | 2073 | 2065 | 865 | 1077 | 484 | 589 | 487 | |
| | 604 | 1067 | 1068 | 760 | 994 | 585 | 495 | 890 | 1885 | 876 | 1285 | 2033 | |
| | 222 | 1762 | 1015 | 801 | 839 | 842 | 1871 | 844 | 435 | 857 | 859 | 650 | |
| | 1237 | 1238 | 861 | 1111 | 2111 | 432 | 694 | 1929 | 314 | 1170 | 1942 | 1908 | |
| | 1560 | 1216 | 1964 | 537 | 311 | 825 | 841 | 1530 | 1002 | 1909 | 443 | 1682 | |
| | 664 | 572 | 2083 | 1070 | 1153 | | | | | | | | |
| 169: | 1320 | 2158 | 541 | 295 | 1150 | 984 | 284 | 743 | 1714 | 1728 | 2008 | 1136 | |
| | 1823 | 847 | 1508 | 2115 | 2130 | 1121 | 1977 | 1802 | 481 | 1466 | 1244 | 1140 | |
| | 717 | 1996 | 1979 | 461 | | | | | | | | | |
| 170: | 806 | 1210 | 871 | 1412 | 248 | 265 | 264 | 246 | 2075 | 286 | 312 | 238 | |
| | 1243 | 744 | 428 | 629 | 1131 | 2148 | 1110 | 1426 | 497 | 933 | 1814 | 1927 | |
| | 1924 | 641 | 1918 | 1578 | 926 | 958 | 1847 | 1197 | 2096 | 939 | 1368 | 1630 | |
| | 1441 | 886 | 490 | 1731 | 1729 | 782 | 557 | 1227 | 1419 | 1179 | 2045 | 1792 | |
| | 1774 | 1186 | 1219 | 1423 | 977 | 1607 | 1791 | 1763 | 1761 | 1789 | 1777 | 682 | |
| | 644 | 268 | 226 | 623 | 829 | 1793 | 1836 | 1265 | 1790 | 263 | 258 | 260 | |
| | 731 | 566 | 1376 | 1720 | 447 | 1579 | 850 | 1953 | 676 | 849 | 1308 | 1869 | |
| | 2092 | 567 | 967 | 2009 | 499 | 936 | 1931 | 403 | 382 | 609 | 680 | 733 | |
| | 982 | 1725 | 1713 | 1711 | 1710 | 1693 | 1214 | | | | | | |
| 171: | 416 | 1799 | 662 | 298 | 1278 | 1126 | 1694 | 1176 | 1187 | 1819 | 2119 | 1397 | |
| | 468 | 1664 | 1493 | 932 | 1393 | 1498 | 2088 | 1811 | 1259 | 1184 | 369 | 1008 | |
| | 1009 | 1966 | 395 | 1036 | 1037 | 1039 | 1033 | 1995 | 1992 | 1040 | 1071 | 1065 | |
| | 1155 | 1141 | 352 | 354 | 1926 | 1826 | 1661 | 1053 | 301 | 1642 | 233 | 1627 | |
| | 2048 | 1593 | 1613 | 1483 | 1667 | 1010 | 437 | 1125 | 1080 | 1092 | 529 | 331 | |
| | 347 | 1982 | 1165 | 1568 | 1542 | 1451 | | | | | | | |
| 172: | 894 | 956 | 2013 | 430 | 1041 | 1448 | 713 | 1021 | 591 | 325 | 786 | 928 | |
| | 1034 | 1522 | 1506 | 1743 | 1905 | 522 | | | | | | | |
| 173: | 203 | 711 | 1833 | 1765 | 1956 | 1295 | 410 | 1050 | 822 | 1496 | 1781 | 762 | |
| | 1095 | 981 | 1006 | 1097 | 1827 | 1611 | 1632 | 559 | 527 | 1512 | 445 | 1473 | |
| 174: | 2086 | 2089 | 2087 | 1061 | 1588 | 1980 | 757 | 1601 | 1581 | 2120 | 2107 | 1679 | |
| | 1818 | 1998 | 1997 | 1959 | 1309 | 1270 | 2056 | 2001 | 2002 | 2003 | 2018 | 1978 | |
| | 761 | 700 | 1271 | 2041 | 1975 | 283 | 291 | 2026 | 1206 | 1363 | 1495 | 1565 | |
| | 1048 | 1537 | 1016 | 2011 | 1989 | 1652 | 561 | 929 | 753 | 1531 | 1515 | 754 | |
| | 631 | 912 | 1687 | 1264 | 200 | 1952 | 2123 | 2135 | 656 | 960 | 381 | 966 | |
| | 2063 | 937 | 545 | 1222 | 1072 | 1392 | 590 | 2150 | 1856 | 1262 | 959 | 925 | |
| | 1589 | 940 | 1591 | 1614 | 811 | 343 | 1797 | 1205 | 571 | 1832 | 877 | 1510 | |
| | 1011 | 944 | 846 | 938 | 948 | 649 | 726 | 897 | 1292 | 1047 | 1944 | 1962 | |
| | 964 | 965 | 1592 | 968 | 1509 | 277 | 1893 | 909 | 1435 | 1648 | 1866 | 1357 | |
| | 1377 | 643 | 2074 | 2072 | 1623 | 1527 | 1957 | 1342 | 1344 | 763 | 1319 | 472 | |
| | 632 | 647 | 633 | 856 | 1353 | 1158 | 1410 | 1046 | 195 | 2166 | 2069 | 2064 | |
| | 907 | 2139 | 1677 | 2159 | 2160 | 2136 | 214 | 232 | 224 | 1845 | 2022 | 2023 | |
| | 1485 | 1487 | 383 | 440 | 1913 | 675 | 1600 | 946 | 274 | 1192 | 1229 | 175 | |
| | 323 | 1335 | | | | | | | | | | | |
| 175: | 2086 | 2089 | 1061 | 1588 | 1349 | 1351 | 1921 | 235 | 1980 | 757 | 1601 | 1581 | |
| | 2120 | 2107 | 1535 | 1538 | 1679 | 1818 | 1998 | 1997 | 1959 | 1309 | 1270 | 2056 | |
| | 1168 | 2001 | 2002 | 2003 | 2018 | 1978 | 700 | 1271 | 2041 | 1975 | 283 | 291 | |
| | 2026 | 2024 | 1206 | 1363 | 1495 | 1565 | 1048 | 1537 | 1016 | 2011 | 1989 | 1652 | |
| | 561 | 929 | 753 | 1634 | 1531 | 1515 | 754 | 1383 | 631 | 912 | 1687 | 1264 | |
| | 200 | 1952 | 2123 | 656 | 960 | 381 | 966 | 2063 | 1222 | 1072 | 1392 | 590 | |
| | 2150 | 1856 | 1262 | 959 | 925 | 1589 | 940 | 1591 | 1614 | 811 | 343 | 1797 | |
| | 1205 | 571 | 1832 | 877 | 1510 | 1011 | 944 | 846 | 938 | 948 | 649 | 726 | |
| | 897 | 1292 | 1047 | 1944 | 1962 | 964 | 1741 | 1592 | 965 | 968 | 1509 | 1590 | |
| | 1341 | 277 | 1893 | 909 | 1435 | 1648 | 1866 | 1377 | 1357 | 643 | 2074 | 2072 | |
| | 1623 | 1527 | 1957 | 1342 | 1344 | 763 | 1319 | 472 | 632 | 647 | 856 | 1353 | |
| | 1158 | 1046 | 2069 | 2064 | 907 | 2139 | 1677 | 2159 | 2160 | 214 | 224 | 232 | |
| | 1845 | 750 | 721 | 2022 | 2023 | 1485 | 1487 | 383 | 440 | 1913 | 441 | 1600 | |
| | 946 | 1335 | 1229 | 174 | 1192 | 274 | | | | | | | |
| 176: | 2086 | 1911 | 1456 | 1447 | 1449 | 751 | 1581 | 2120 | 1780 | 1431 | 208 | 1998 | |
| | 1997 | 1959 | 1306 | 2056 | 1168 | 2001 | 2003 | 2018 | 1978 | 700 | 1465 | 1444 | |
| | 1461 | 1429 | 1428 | 1967 | 1912 | 1271 | 2041 | 1215 | 1439 | 1457 | 1442 | 1016 | |
| | 1634 | 1585 | 596 | 631 | 215 | 217 | 200 | 1952 | 2123 | 656 | 950 | 960 | |
| | 207 | 209 | 381 | 2027 | 1671 | 749 | 1258 | 2063 | 1222 | 1195 | 1551 | 1902 | |
| | 462 | 194 | 1392 | 450 | 1424 | 1407 | 1408 | 1558 | 1109 | 959 | 925 | 1591 | |
| | 940 | 1385 | 1382 | 1614 | 494 | 2116 | 1815 | 877 | 1510 | 944 | 1460 | 771 | |
| | 783 | 770 | 1274 | 938 | 1387 | 1275 | 1988 | 580 | 1626 | 2153 | 964 | 1592 | |
| | 965 | 968 | 1787 | 418 | 1719 | 1230 | 460 | 1619 | 1232 | 1305 | 297 | 1494 | |
| | 2059 | 1377 | 1333 | 1948 | 517 | 643 | 691 | 2074 | 1844 | 1410 | 2166 | 1643 | |
| | 907 | 2139 | 954 | 2022 | 2023 | 1490 | 1406 | 1427 | 946 | 2137 | | | |
| 177: | 303 | 408 | 860 | 2124 | 373 | 1812 | 795 | 729 | 814 | 2015 | | | |
| 178: | 781 | 1325 | 2061 | 1350 | 1348 | 1346 | 467 | 903 | 915 | 1759 | 1882 | 1685 | |
| | 1688 | 808 | 2117 | 2094 | 544 | 1547 | 1770 | 1566 | 1174 | 920 | 1984 | 2036 | |
| | 470 | 319 | 1288 | 1290 | 1375 | 1395 | 1296 | 1330 | 1328 | 1311 | 342 | 1758 | |
| | 1723 | 292 | 1532 | 2044 | 990 | 1112 | 764 | 668 | 1063 | 1123 | 1969 | 542 | |
| | 916 | 945 | 1367 | 1364 | 1313 | 1314 | 1324 | 1355 | 1352 | 1293 | 1294 | 1378 | |
| | 1401 | 1399 | 1310 | 2082 | 2068 | 1597 | 1160 | 1773 | 1529 | 2025 | | | |
| 179: | 303 | 408 | 860 | 2124 | 373 | 1812 | 795 | 729 | 814 | 2015 | | | |
| 180: | 1754 | 508 | 469 | 1907 | 1965 | 595 | 1464 | 1458 | 400 | 514 | 1617 | 1810 | |
| | 1839 | 565 | 348 | 366 | 389 | 391 | 431 | 1825 | 1004 | | | | |
| 181: | 1919 | 972 | 351 | 349 | 1188 | 2164 | 1749 | 305 | 287 | 1329 | 983 | 935 | |
| | 1876 | 780 | 1747 | 1883 | 1175 | 1454 | 1326 | 2161 | 1042 | 1920 | 1654 | 843 | |
| | 1717 | 2014 | 900 | 824 | 820 | 530 | 1653 | | | | | | |
| 182: | 1225 | 666 | 2099 | 1453 | 1467 | 774 | 1337 | 1137 | 562 | 2080 | 1235 | 1574 | |
| | 1963 | 1518 | 598 | 670 | 1462 | 719 | 442 | 523 | 1443 | 947 | 2000 | 1445 | |
| | 2054 | 1675 | 931 | 706 | 584 | 1240 | 718 | 838 | 1764 | 2114 | 2109 | 1480 | |
| | 1772 | 1022 | 1171 | 332 | | | | | | | | | |
| 183: | 1528 | 1786 | 1660 | 387 | 502 | 1562 | 204 | 1007 | 992 | 1880 | 1073 | 788 | |
| | 686 | 1388 | 1267 | 1840 | 2019 | 2050 | 1938 | 1943 | 1546 | 872 | 1024 | 485 | |
| | 1934 | 1622 | 1629 | 1541 | 340 | 674 | 1477 | 742 | 511 | 922 | 231 | 1960 | |
| | 2062 | 1440 | 1689 | 1051 | 2122 | 1755 | 583 | 2156 | 2162 | 188 | 2127 | 1481 | |
| | 1563 | 1897 | 672 | 394 | 225 | 784 | 1526 | 448 | 1795 | 302 | 1862 | 2028 | 1983 |
| | 646 | 304 | 310 | 739 | 1459 | | | | | | | | |
| 184: | 1754 | 508 | 469 | 1907 | 1965 | 595 | 1464 | 1458 | 400 | 514 | 1617 | 1810 | |
| | 1839 | 565 | 348 | 366 | 389 | 391 | 431 | 1825 | 1004 | | | | |
| 185: | 2016 | 213 | 1056 | 254 | 799 | 1217 | 600 | 848 | 334 | 1134 | 444 | 1343 | |
| | 1124 | 1923 | 904 | 812 | 953 | 985 | 1587 | 1000 | 999 | 716 | 1877 | 828 | |
| | 372 | 1162 | 854 | 934 | 599 | 884 | 1887 | 115 | 621 | 251 | 1091 | 1768 | |
| | 116 | 2155 | | | | | | | | | | | |
| 186: | 1144 | 1143 | 1813 | 386 | 995 | 502 | 1668 | 1007 | 1279 | 271 | 1651 | 1024 | |
| | 1541 | 356 | 1540 | 660 | 1689 | 1051 | 488 | 2121 | 555 | 683 | 1987 | 581 | |
| | 132 | 131 | 130 | 129 | 128 | 973 | 672 | 438 | 1272 | 1666 | 1384 | 1520 | |
| | 698 | 1173 | 1513 | 336 | 1269 | 756 | 943 | 1949 | 776 | 243 | 1241 | 1970 | |
| | 646 | 304 | 402 | | | | | | | | | | |
| 187: | 269 | 1470 | 1317 | 255 | 976 | 320 | 957 | 1849 | 1365 | 1760 | 577 | 1968 | |
| | 1161 | 576 | 777 | 308 | | | | | | | | | |
| 188: | 502 | 1562 | 1007 | 992 | 1073 | 1880 | 788 | 686 | 1840 | 2019 | 1938 | 1267 | |
| | 1388 | 2050 | 1943 | 1546 | 872 | 663 | 1024 | 485 | 1629 | 1622 | 674 | 1477 | |
| | 340 | 511 | 742 | 922 | 231 | 1130 | 1202 | 1226 | 1440 | 1689 | 1051 | 2122 | |
| | 1755 | 583 | 1266 | 1563 | 672 | 1868 | 784 | 346 | 802 | 309 | 2076 | 358 | |
| | 1146 | 475 | 1575 | 199 | 536 | 1420 | 1421 | 1394 | 310 | 739 | 183 | 1062 | |
| | 1459 | 2133 | 2157 | 409 | 625 | 689 | 646 | 304 | 2162 | | | | |
| 189: | 1163 | 896 | 1164 | 1850 | 337 | 341 | 1032 | 874 | 1182 | 230 | 307 | 540 | |
| | 626 | 1374 | 1045 | 728 | 746 | 2113 | 2142 | 533 | 951 | 980 | 1043 | 1784 | |
| | 845 | 1650 | 1925 | 498 | 515 | 1404 | 1930 | 642 | | | | | |
| 190: | 1919 | 972 | 351 | 349 | 1188 | 2164 | 1749 | 305 | 287 | 1329 | 983 | 935 | |
| | 1876 | 780 | 1747 | 1883 | 1175 | 1454 | 1326 | 2161 | 1042 | 1920 | 1654 | 843 | |
| | 1717 | 2014 | 900 | 824 | 820 | 530 | 1653 | | | | | | |

### Example 7

### Selection of Transgenic Plants with Enhanced Agronomic Trait(s)

This example illustrates identification of transgenic plant cells of the invention by screening derived plants and seeds for enhanced trait. Transgenic seed and plants in corn, soybean, cotton or canola with recombinant DNA constructs identified in Table 1 are prepared by plant cells transformed with DNA that is stably integrated into a chromosome of the plant cell. Progeny transgenic plants and seed of the transformed plant cells are screened for enhanced water use efficiency, enhanced cold tolerance, increased yield, enhanced nitrogen use efficiency, enhanced seed protein and enhanced seed oil as compared to control plants

### A. Selection for Enhanced Nitrogen Use Efficiency (NUE)

Transgenic corn seeds provided by the present invention are planted in fields with three levels of nitrogen (N) fertilizer being applied, i.e. low level (0 N), medium level (80 lb/ac) and high level (180 lb/ac). A variety of physiological traits are monitored. Plants with enhanced NUE provide higher yield as compared to control plants.

### B. Selection for Increased Yield

Effective selection of enhanced yielding transgenic plants uses hybrid progeny of the transgenic plants for corn, cotton, and canola, or inbred progeny of transgenic plants for soybean, canola and cotton over multiple locations with plants grown under optimal production management practices, and maximum pest control. A useful target for improved yield is a 5% to 10% increase as compared to yield produced by plants grown from seed for a control plant. Selection methods may be applied in multiple and diverse geographic locations, for example up to 16 or more locations, over one or more planting seasons, for example at least two planting seasons, to statistically distinguish yield improvement from natural environmental effects.

### C. Selection for Enhanced Water Use Efficiency (WUE)

The selection process imposes a water withholding period to induce drought stress followed by watering. For example, for corn, a useful selection process imposes 3 drought/re-water cycles on plants over a total period of 15 days after an initial stress free growth period of 11 days. Each cycle consists of 5 days, with no water being applied for the first four days and a water quenching on the 5th day of the cycle. The primary phenotypes analyzed by the selection method are the changes in plant growth rate as determined by height and biomass during a vegetative drought treatment.

### D. Selection for Growth Under Cold Stress

(1) Cold germination assay—Trays of transgenic and control seeds are placed in a growth chamber at 9.7° C. for 24 days (no light). Seeds having higher germination rates as compared to the control are identified.

(2) Cold field efficacy trial—A cold field efficacy trial is used to identify recombinant DNA constructs that confer enhanced cold vigor at germination and early seedling growth under early spring planting field conditions in conventional-till and simulated no-till environments. Seeds are planted into the ground around two weeks before local farmers begin to plant corn so that a significant cold stress is exerted onto the crop, named as cold treatment. Seeds also are planted under local optimal planting conditions such that the crop has little or no exposure to cold condition, named as normal treatment. At each location, seeds are planted under both cold and normal conditions with 3 repetitions per treatment. Two temperature monitors are set up at each location to monitor both air and soil temperature daily.

Seed emergence is defined as the point when the growing shoot breaks the soil surface. The number of emerged seedlings in each plot is counted everyday from the day the earliest plot begins to emerge until no significant changes in emergence occur. In addition, for each planting date, the latest date when emergence is 0 in all plots is also recorded. Seedling vigor is also rated at V3-V4 stage before the average of corn plant height reaches 10 inches, with 1=excellent early growth, 5=Average growth and 9=poor growth. Days to 50% emergence, maximum percent emergence and seedling vigor are used to determine plants with enhanced cold tolerance.

E. Screens for Transgenic Plant Seeds with Increased Protein and/or Oil Levels

This example sets forth a high-throughput selection for identifying plant seeds with improvement in seed composition using the Infratec 1200 series Grain Analyzer, which is a near-infrared transmittance spectrometer used to determine the composition of a bulk seed sample (Table 9). Near infrared analysis is a non-destructive, high-throughput method that can analyze multiple traits in a single sample scan. An NIR calibration for the analytes of interest is used to predict the values of an unknown sample. The NIR spectrum is obtained for the sample and compared to the calibration using a complex chemometric software package that provides predicted values as well as information on how well the sample fits in the calibration.

Infratec Model 1221, 1225, or 1227 with transport module by Foss North America is used with cuvette, item #1000-4033, Foss North America or for small samples with small cell cuvette, Foss standard cuvette modified by Leon Girard Co. Corn and soy check samples of varying composition maintained in check cell cuvettes are supplied by Leon Girard Co. NIT collection software is provided by Maximum Consulting Inc Software. Calculations are performed automatically by the software. Seed samples are received in packets or containers with barcode labels from the customer. The seed is poured into the cuvettes and analyzed as received.

TABLE 9

| | |
|---|---|
| Typical sample(s): | Whole grain corn and soybean seeds |
| Analytical time to run method: | Less than 0.75 min per sample |
| Total elapsed time per run: | 1.5 minute per sample |
| Typical and minimum sample | Corn typical: 50 cc; minimum 30 cc |
| size: | Soybean typical: 50 cc; minimum 5 cc |
| Typical analytical range: | Determined in part by the specific |
| | calibration. |
| | Corn - moisture 5-15%, oil 5-20%, |
| | protein 5-30%, starch 50-75%, and |
| | density 1.0-1.3%. |
| | Soybean - moisture 5-15%, oil 15- |
| | 25%, and protein 35-50%. |

### Example 8

### Consensus Sequence

This example illustrates the identification of consensus amino acid sequence for the proteins and homologs encoded by DNA that is used to prepare the transgenic seed and plants of this invention having enhanced agronomic traits.

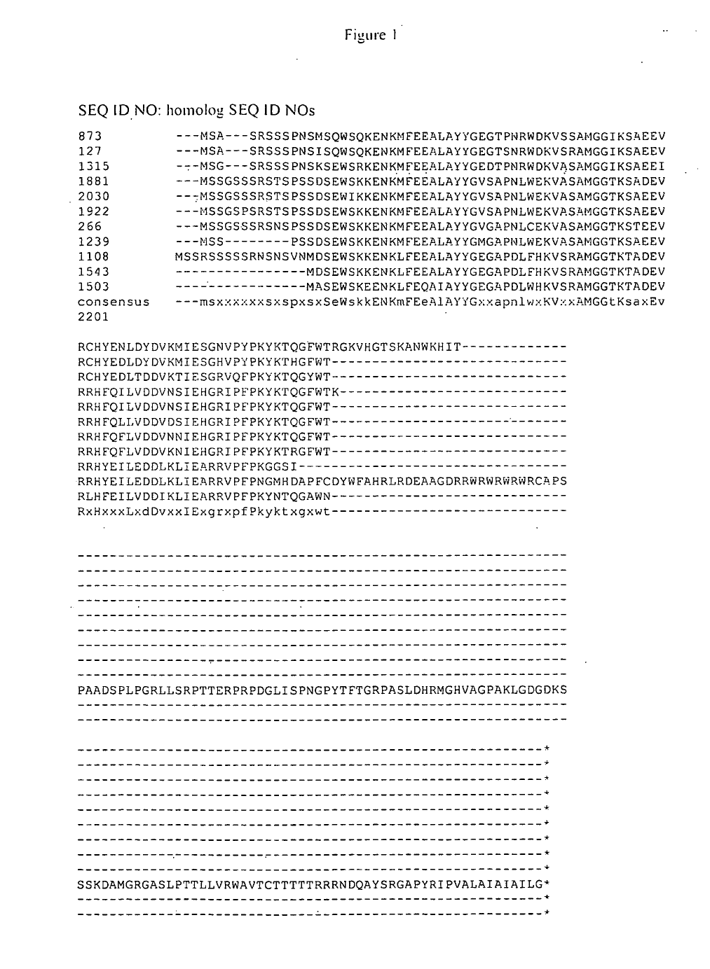

ClustalW program was selected for multiple sequence alignments of the amino acid sequence of SEQ ID NO: 127 and its 10 homologs. Three major factors affecting the sequence alignments dramatically are (1) protein weight matrices; (2) gap open penalty; (3) gap extension penalty. Protein weight matrices available for ClustalW program include Blosum, Pam and Gonnet series. Those parameters with gap open penalty and gap extension penalty were extensively tested. On the basis of the test results, Blosum weight matrix, gap open penalty of 10 and gap extension penalty of 1 were chosen for multiple sequence alignment. FIG. 1 shows the sequences of SEQ ID NO: 127, its homologs and the consensus sequence (SEQ ID NO: 2201) at the end. The symbols for consensus sequence are (1) uppercase letters for 100% identity in all positions of multiple sequence alignment output; (2) lowercase letters for >=70% identity; symbol; (3) “X” indicated <70% identity; (4) dashes “-” meaning that gaps were in >=70% sequences.

The consensus amino acid sequence can be used to identify DNA corresponding to the full scope of this invention that is useful in providing transgenic plants, for example corn and soybean plants with enhanced agronomic traits, for example improved nitrogen use efficiency, improved yield, improved water use efficiency and/or improved growth under cold stress, due to the expression in the plants of DNA encoding a protein with amino acid sequence identical to the consensus amino acid sequence.

### Example 9

### Identification of Amino Acid Domain by Pfam Analysis

This example illustrates the identification of protein domain and domain module by Pfam analysis.

The amino acid sequence of the expressed proteins that are shown to be associated with an enhanced trait were analyzed for Pfam protein family against the current Pfam collection of multiple sequence alignments and Hidden Markov models using the HMMER software in the appended computer listing. The Pfam protein domains and modules for the proteins for the proteins of SEQ ID NO: 96 through 193 are shown in Tables 11 and 10 respectively. The Hidden Markov model databases for the identified pfam domains are also in the appended computer listing allowing identification of other homologous proteins and their cognate encoding DNA to enable the full breadth of the invention for a person of ordinary skill in the art. Certain proteins are identified by a single Pfam domain and others by multiple Pfam domains. For instance, the protein with amino acids of SEQ ID NO: 98 is characterized by three Pfam domains, i.e. KNOX1, KNOX2 and ELK.

TABLE 10
Pfam domain module annotation

| PEP SEQ | | | |
|---|---|---|---|
| ID NO | Gene ID | Pfam domain module | Position |
| 154 | PHE0001067_8154.pep | Homeobox | 97-158 |
| 106 | PHE0002062_5913.pep | Myb_DNA- | 14-61::67-112 |
| | | binding::Myb_DNA-binding | |
| 140 | PHE0002149_7487.pep | Myb_DNA-binding | 24-69 |
| 171 | PHE0002149_8748.pep | Myb_DNA-binding | 24-69 |
| 107 | PHE0002531_5926.pep | zf-Dof | 39-101 |
| 152 | PHE0002531_7985.pep | zf-Dof | 39-101 |
| 151 | PHE0003473_7927.pep | zf-C2H2::zf-C2H2 | 72-94::149-171 |
| 138 | PHE0003664_7436.pep | AP2 | 21-84 |
| 166 | PHE0003664_8637.pep | AP2 | 21-84 |
| 137 | PHE0003673_7430.pep | Response_reg::Myb_DNA- | 13-126::197-247 |
| | | binding | |
| 188 | PHE0004332_PMON95104.pep | AP2 | 104-168 |
| 187 | PHE0004356_PMON93862.pep | B3 | 23-128 |
| 183 | PHE0004357_PMON94163.pep | AP2 | 135-199 |
| 153 | PHE0004463_8059.pep | AP2 | 6-69 |
| 186 | PHE0004463_PMON94432.pep | AP2 | 6-69 |
| 185 | PHE0004624_PMON94400.pep | B3::Auxin_resp::AUX_IAA | 141-246::268-350::640-805 |
| 96 | PHE0004633_5508.pep | HLH | 104-152 |
| 189 | PHE0004644_PMON95096.pep | HLH | 327-374 |
| 184 | PHE0004646_PMON94352.pep | NAM | 17-139 |
| 180 | PHE0004646_PMON94356.pep | NAM | 17-139 |
| 182 | PHE0004648_PMON95051.pep | B3::B3 | 13-105::148-244 |
| 181 | PHE0004723_PMON94660.pep | AUX_IAA | 6 |
| 190 | PHE0004723_PMON95121.pep | AUX_IAA | 6 |
| 97 | PHE0004738_5674.pep | NAM | 96 |
| 98 | PHE0004814_5801.pep | KNOX1::KNOX2::ELK | 38-82::92-147::203-244 |
| 133 | PHE0004816_7303.pep | HLH | 19-68 |
| 136 | PHE0004816_7418.pep | HLH | 19-68 |
| 135 | PHE0004816_7421.pep | HLH | 19-68 |
| 139 | PHE0004816_7445.pep | HLH | 19-68 |
| 99 | PHE0004817_5809.pep | HLH | 20-69 |
| 100 | PHE0004817_5810.pep | HLH | 20-69 |
| 103 | PHE0004817_5901.pep | HLH | 20-69 |
| 101 | PHE0004821_5819.pep | HLH | 105-154 |
| 102 | PHE0004828_5826.pep | HLH | 107-156 |
| 104 | PHE0004861_5910.pep | GRAS | 26-325 |
| 105 | PHE0004863_5912.pep | AT_hook::AT_HOOK::DUF296 | 121-133 |
| 119 | PHE0004877_7030.pep | Response_reg::Myb_DNA- | 13-126::197-247 |
| | | binding | |
| 108 | PHE0004914_5971.pep | Myb_DNA- | 11-60::117-164 |
| | | binding::Myb_DNA-binding | |
| 109 | PHE0004924_5982.pep | TCP | 38-253 |
| 110 | PHE0004925_5983.pep | SBP | 58-136 |
| 111 | PHE0004938_5994.pep | GRAS | 154-454 |
| 159 | PHE0004938_8370.pep | GRAS | 154-454 |
| 112 | PHE0004957_6019.pep | zf-C2H2 | 68-90 |
| 113 | PHE0004958_6020.pep | zf-Dof | 104-166 |
| 114 | PHE0004959_6021.pep | AP2 | 128-191 |
| 115 | PHE0004974_6040.pep | B3::Auxin_resp | 148-253 |
| 116 | PHE0004975_6041.pep | B3::Auxin_resp | 136-241::263-345 |
| 117 | PHE0004987_6056.pep | Myb_DNA-binding | 21-72 |
| 173 | PHE0004987_8771.pep | Myb_DNA-binding | 21-72 |
| 118 | PHE0005005_7034.pep | Myb_DNA-binding | 96-143 |
| 124 | PHE0006004_7082.pep | NAM | 18-147 |
| 167 | PHE0006004_8667.pep | NAM | 18-147 |
| 125 | PHE0006022_7105.pep | EIN3 | 30-426 |
| 168 | PHE0006022_8690.pep | EIN3 | 30-426 |
| 126 | PHE0006023_7240.pep | HLH | 160-210 |
| 172 | PHE0006023_8762.pep | HLH | 160-210 |
| 120 | PHE0006057_7048.pep | HLH | 12-61 |
| 121 | PHE0006057_7053.pep | HLH | 12-61 |
| 150 | PHE0006057_7929.pep | HLH | 12-61 |
| 122 | PHE0006070_7067.pep | bZIP_2 | 96-153 |
| 123 | PHE0006073_7072.pep | bZIP_2 | 96-153 |
| 128 | PHE0006237_7261.pep | AP2 | 6-69 |
| 130 | PHE0006237_7268.pep | AP2 | 6-69 |
| 129 | PHE0006237_7274.pep | AP2 | 6-69 |
| 131 | PHE0006237_7277.pep | AP2 | 6-69 |
| 132 | PHE0006237_7284.pep | AP2 | 6-69 |
| 141 | PHE0006290_7498.pep | zf-B_box::zf-B_box | 1-47::48-90 |
| 169 | PHE0006290_8689.pep | zf-B_box::zf-B_box | 1-47::48-90::355-393 |
| 134 | PHE0006291_7319.pep | zf-B_box::CCT | 3-50::309-347 |
| 155 | PHE0006350_8201.pep | GRAS | 98-403 |
| 143 | PHE0006384_7737.pep | Myb_DNA- | 14-61::67-112 |
| | | binding::Myb_DNA-binding | |
| 144 | PHE0006384_7789.pep | Myb_DNA- | 14-61::67-112 |
| | | binding::Myb_DNA-binding | |
| 147 | PHE0006384_7839.pep | Myb_DNA- | 14-61::67-112 |
| | | binding::Myb_DNA-binding | |
| 142 | PHE0006423_7664.pep | Myb_DNA- | 14-61-67-112 |
| | | binding::Myb_DNA-binding | |
| 170 | PHE0006423_8696.pep | Myb_DNA- | 14-61::67-112 |
| | | binding::Myb_DNA-binding | |
| 148 | PHE0006448_7859.pep | RWP-RK::PB1 | 553-604::741-823 |
| 149 | PHE0006504_7876.pep | F-box::TUB | 49-104::115-424 |
| 145 | PHE0006507_7828.pep | CBFD_NFYB_HMF | 1-40 |
| 146 | PHE0006509_7846.pep | SBP | 64-142 |
| 158 | PHE0006527_8369.pep | CBFD_NFYB_HMF | 26-91 |
| 157 | PHE0006546_8310.pep | Response_reg::Myb_DNA- | 28-141::225-275 |
| | | binding | |
| 156 | PHE0006605_8233.pep | GATA | 223-258 |
| 163 | PHE0006752_8521.pep | SRF-TF | 9-59 |
| 163 | PHE0006752_8521.pep | K-box | 75-174 |
| 160 | PHE0006774_8489.pep | CBFD_NFYB_HMF | 34-106 |
| 161 | PHE0006778_8503.pep | CBFD_NFYB_HMF | 34-106 |
| 164 | PHE0006779_8565.pep | CBFD_NFYB_HMF | 34-106 |
| 162 | PHE0006780_8502.pep | CBFD_NFYB_HMF | 34-106 |
| 165 | PHE0006781_8573.pep | CBFD_NFYB_HMF | 34-106 |
| 174 | PHE0006858_8859.pep | SRF-TF::K-box | 9-59::75-174 |
| 175 | PHE0006860_8863.pep | SRF-TF::K-box | 9-59::75-174 |
| 177 | PHE0006951_9137.pep | zf-C2H2 | 152-175 |
| 179 | PHE0006951_9173.pep | zf-C2H2 | 152-175 |
| 176 | PHE0006955_9129.pep | SRF-TF::K-box | 9-59::85-175 |
| 178 | PHE0006981_9158.pep | GRAS | 149-456 |

TABLE 11
Pfam domain annotation

| PEP | | | | | | |
|---|---|---|---|---|---|---|
| SEQ | | | | | | |
| ID | | | | | | |
| NO | GENE ID | Pfam domain name | Begin | Stop | Score | E-value |
| 154 | PHE0001067_8154.pep | Homeobox | 97 | 158 | 68 | 2.70E−17 |
| 106 | PHE0002062_5913.pep | Myb_DNA-binding | 14 | 61 | 44.5 | 3.20E−10 |
| 106 | PHE0002062_5913.pep | Myb_DNA-binding | 67 | 112 | 47.8 | 3.20E−11 |
| 140 | PHE0002149_7487.pep | Myb_DNA-binding | 24 | 69 | 54.4 | 3.40E−13 |
| 171 | PHE0002149_8748.pep | Myb_DNA-binding | 24 | 69 | 54.4 | 3.40E−13 |
| 107 | PHE0002531_5926.pep | zf-Dof | 39 | 101 | 133.7 | 4.60E−37 |
| 152 | PHE0002531_7985.pep | zf-Dof | 39 | 101 | 133.7 | 4.60E−37 |
| 151 | PHE0003473_7927.pep | zf-C2H2 | 72 | 94 | 25.6 | 0.00016 |
| 151 | PHE0003473_7927.pep | zf-C2H2 | 149 | 171 | 20.5 | 0.0055 |
| 138 | PHE0003664_7436.pep | AP2 | 21 | 84 | 135.2 | 1.60E−37 |
| 166 | PHE0003664_8637.pep | AP2 | 21 | 84 | 135.2 | 1.60E−37 |
| 137 | PHE0003673_7430.pep | Response_reg | 13 | 126 | 104.9 | 2.20E−28 |
| 137 | PHE0003673_7430.pep | Myb_DNA-binding | 197 | 247 | 46.4 | 8.90E−11 |
| 188 | PHE0004332_PMON95104.pep | AP2 | 104 | 168 | 156.7 | 5.40E−44 |
| 187 | PHE0004356_PMON93862.pep | B3 | 23 | 128 | 64.1 | 4.20E−16 |
| 183 | PHE0004357_PMON94163.pep | AP2 | 135 | 199 | 150.2 | 5.10E−42 |
| 153 | PHE0004463_8059.pep | AP2 | 6 | 69 | 116.5 | 7.10E−32 |
| 186 | PHE0004463_PMON94432.pep | AP2 | 6 | 69 | 116.5 | 7.10E−32 |
| 185 | PHE0004624_PMON94400.pep | B3 | 141 | 246 | 110.7 | 4.00E−30 |
| 185 | PHE0004624_PMON94400.pep | Auxin_resp | 268 | 350 | 198.6 | 1.30E−56 |
| 185 | PHE0004624_PMON94400.pep | AUX_IAA | 640 | 805 | −72.2 | 0.00025 |
| 96 | PHE0004633_5508.pep | HLH | 104 | 152 | 39.4 | 1.10E−08 |
| 189 | PHE0004644_PMON95096.pep | HLH | 327 | 374 | 36.7 | 7.20E−08 |
| 184 | PHE0004646_PMON94352.pep | NAM | 17 | 139 | 58.4 | 2.20E−14 |
| 180 | PHE0004646_PMON94356.pep | NAM | 17 | 139 | 58.4 | 2.20E−14 |
| 182 | PHE0004648_PMON95051.pep | B3 | 13 | 105 | 117 | 4.90E−32 |
| 182 | PHE0004648_PMON95051.pep | B3 | 148 | 244 | 110.3 | 5.20E−30 |
| 181 | PHE0004723_PMON94660.pep | AUX_IAA | 6 | 173 | 339.7 | 4.50E−99 |
| 190 | PHE0004723_PMON95121.pep | AUX_IAA | 6 | 173 | 339.7 | 4.50E−99 |
| 97 | PHE0004738_5674.pep | NAM | 96 | 239 | 184.9 | 1.80E−52 |
| 98 | PHE0004814_5801.pep | KNOX1 | 38 | 82 | 63.3 | 7.00E−16 |
| 98 | PHE0004814_5801.pep | KNOX2 | 92 | 147 | 83.5 | 6.00E−22 |
| 98 | PHE0004814_5801.pep | ELK | 203 | 224 | 34.1 | 4.50E−07 |
| 133 | PHE0004816_7303.pep | HLH | 19 | 68 | 62.5 | 1.30E−15 |
| 136 | PHE0004816_7418.pep | HLH | 19 | 68 | 62.5 | 1.30E−15 |
| 135 | PHE0004816_7421.pep | HLH | 19 | 68 | 62.5 | 1.30E−15 |
| 139 | PHE0004816_7445.pep | HLH | 19 | 68 | 62.5 | 1.30E−15 |
| 99 | PHE0004817_5809.pep | HLH | 20 | 69 | 57.6 | 3.70E−14 |
| 100 | PHE0004817_5810.pep | HLH | 20 | 69 | 57.6 | 3.70E−14 |
| 103 | PHE0004817_5901.pep | HLH | 20 | 69 | 57.6 | 3.70E−14 |
| 101 | PHE0004821_5819.pep | HLH | 105 | 154 | 61.6 | 2.40E−15 |
| 102 | PHE0004828_5826.pep | HLH | 107 | 156 | 60 | 7.00E−15 |
| 104 | PHE0004861_5910.pep | GRAS | 26 | 325 | 369.5 | 4.70E−108 |
| 105 | PHE0004863_5912.pep | AT_hook | 121 | 133 | 17.5 | 0.02 |
| 105 | PHE0004863_5912.pep | AT_hook | 182 | 194 | 12.6 | 0.14 |
| 105 | PHE0004863_5912.pep | DUF296 | 212 | 332 | 177.3 | 3.40E−50 |
| 119 | PHE0004877_7030.pep | Response_reg | 13 | 126 | 104.9 | 2.20E−28 |
| 119 | PHE0004877_7030.pep | Myb_DNA-binding | 197 | 247 | 46.4 | 8.90E−11 |
| 108 | PHE0004914_5971.pep | Myb_DNA-binding | 11 | 60 | 22.4 | 0.0014 |
| 108 | PHE0004914_5971.pep | Myb_DNA-binding | 117 | 164 | 49.3 | 1.20E−11 |
| 109 | PHE0004924_5982.pep | TCP | 38 | 253 | 139.2 | 1.00E−38 |
| 110 | PHE0004925_5983.pep | SBP | 58 | 136 | 173.4 | 5.30E−49 |
| 111 | PHE0004938_5994.pep | GRAS | 154 | 454 | 524.3 | 1.20E−154 |
| 159 | PHE0004938_8370.pep | GRAS | 154 | 454 | 524.3 | 1.20E−154 |
| 112 | PHE0004957_6019.pep | zf-C2H2 | 68 | 90 | 21.6 | 0.0026 |
| 113 | PHE0004958_6020.pep | zf-Dof | 104 | 166 | 140.5 | 4.20E−39 |
| 114 | PHE0004959_6021.pep | AP2 | 128 | 191 | 141.1 | 2.70E−39 |
| 115 | PHE0004974_6040.pep | B3 | 148 | 253 | 114.1 | 3.60E−31 |
| 115 | PHE0004974_6040.pep | Auxin_resp | 275 | 357 | 156.9 | 4.80E−44 |
| 115 | PHE0004974_6040.pep | AUX_IAA | 623 | 809 | −63.7 | 6.00E−05 |
| 116 | PHE0004975_6041.pep | B3 | 136 | 241 | 113.4 | 6.10E−31 |
| 116 | PHE0004975_6041.pep | Auxin_resp | 263 | 345 | 170 | 5.40E−48 |
| 117 | PHE0004987_6056.pep | Myb_DNA-binding | 21 | 72 | 48.3 | 2.30E−11 |
| 173 | PHE0004987_8771.pep | Myb_DNA-binding | 21 | 72 | 48.3 | 2.30E−11 |
| 118 | PHE0005005_7034.pep | Myb_DNA-binding | 96 | 143 | 54 | 4.60E−13 |
| 124 | PHE0006004_7082.pep | NAM | 18 | 147 | 257.9 | 1.90E−74 |
| 167 | PHE0006004_8667.pep | NAM | 18 | 147 | 257.9 | 1.90E−74 |
| 125 | PHE0006022_7105.pep | EIN3 | 30 | 426 | 983.5 | 7.20E−293 |
| 168 | PHE0006022_8690.pep | EIN3 | 30 | 426 | 983.5 | 7.20E−293 |
| 126 | PHE0006023_7240.pep | HLH | 160 | 210 | 36.8 | 6.80E−08 |
| 172 | PHE0006023_8762.pep | HLH | 160 | 210 | 36.8 | 6.80E−08 |
| 120 | PHE0006057_7048.pep | HLH | 12 | 61 | 60.5 | 5.10E−15 |
| 121 | PHE0006057_7053.pep | HLH | 12 | 61 | 59.8 | 8.10E−15 |
| 150 | PHE0006057_7929.pep | HLH | 12 | 61 | 60.5 | 5.10E−15 |
| 122 | PHE0006070_7067.pep | bZIP_2 | 96 | 153 | 65.7 | 1.30E−16 |
| 122 | PHE0006070_7067.pep | bZIP_1 | 96 | 156 | 18.3 | 0.0014 |
| 123 | PHE0006073_7072.pep | bZIP_1 | 96 | 156 | 18.3 | 0.0014 |
| 123 | PHE0006073_7072.pep | bZIP_2 | 96 | 153 | 65.7 | 1.30E−16 |
| 128 | PHE0006237_7261.pep | AP2 | 6 | 69 | 121.7 | 1.90E−33 |
| 130 | PHE0006237_7268.pep | AP2 | 6 | 69 | 121.7 | 1.90E−33 |
| 129 | PHE0006237_7274.pep | AP2 | 6 | 69 | 121.7 | 1.90E−33 |
| 131 | PHE0006237_7277.pep | AP2 | 6 | 69 | 121.7 | 1.90E−33 |
| 132 | PHE0006237_7284.pep | AP2 | 6 | 69 | 121.7 | 1.90E−33 |
| 141 | PHE0006290_7498.pep | zf-B_box | 1 | 47 | 44.6 | 3.00E−10 |
| 141 | PHE0006290_7498.pep | zf-B_box | 48 | 90 | 23.5 | 0.00039 |
| 141 | PHE0006290_7498.pep | CCT | 355 | 393 | 72.3 | 1.40E−18 |
| 169 | PHE0006290_8689.pep | zf-B_box | 1 | 47 | 44.6 | 3.00E−10 |
| 169 | PHE0006290_8689.pep | zf-B_box | 48 | 90 | 23.5 | 0.00039 |
| 169 | PHE0006290_8689.pep | CCT | 355 | 393 | 72.3 | 1.40E−18 |
| 134 | PHE0006291_7319.pep | zf-B_box | 3 | 50 | 56.9 | 6.10E−14 |
| 134 | PHE0006291_7319.pep | CCT | 309 | 347 | 69.6 | 9.10E−18 |
| 155 | PHE0006350_8201.pep | GRAS | 98 | 403 | 400 | 3.20E−117 |
| 143 | PHE0006384_7737.pep | Myb_DNA-binding | 14 | 61 | 43.1 | 8.70E−10 |
| 143 | PHE0006384_7737.pep | Myb_DNA-binding | 67 | 112 | 50 | 7.30E−12 |
| 144 | PHE0006384_7789.pep | Myb_DNA-binding | 14 | 61 | 43.1 | 8.70E−10 |
| 144 | PHE0006384_7789.pep | Myb_DNA-binding | 67 | 112 | 50 | 7.30E−12 |
| 147 | PHE0006384_7839.pep | Myb_DNA-binding | 14 | 61 | 43.1 | 8.70E−10 |
| 147 | PHE0006384_7839.pep | Myb_DNA-binding | 67 | 112 | 50 | 7.30E−12 |
| 142 | PHE0006423_7664.pep | Myb_DNA-binding | 14 | 61 | 51.6 | 2.50E−12 |
| 142 | PHE0006423_7664.pep | Myb_DNA-binding | 67 | 112 | 35.1 | 2.20E−07 |
| 170 | PHE0006423_8696.pep | Myb_DNA-binding | 14 | 61 | 51.6 | 2.50E−12 |
| 170 | PHE0006423_8696.pep | Myb_DNA-binding | 67 | 112 | 35.1 | 2.20E−07 |
| 148 | PHE0006448_7859.pep | RWP-RK | 553 | 604 | 110.7 | 3.80E−30 |
| 148 | PHE0006448_7859.pep | PB1 | 741 | 823 | 92.6 | 1.10E−24 |
| 149 | PHE0006504_7876.pep | F-box | 49 | 104 | 29.5 | 1.10E−05 |
| 149 | PHE0006504_7876.pep | Tub | 115 | 424 | 691.2 | 7.00E−205 |
| 145 | PHE0006507_7828.pep | CBFD_NFYB_HMF | 1 | 40 | 31.5 | 2.80E−06 |
| 146 | PHE0006509_7846.pep | SBP | 64 | 142 | 188.1 | 1.90E−53 |
| 158 | PHE0006527_8369.pep | CBFD_NFYB_HMF | 26 | 91 | 130.9 | 3.20E−36 |
| 157 | PHE0006546_8310.pep | Response_reg | 28 | 141 | 92.2 | 1.40E−24 |
| 157 | PHE0006546_8310.pep | Myb_DNA-binding | 225 | 275 | 45.6 | 1.50E−10 |
| 156 | PHE0006605_8233.pep | GATA | 223 | 258 | 67.8 | 3.20E−17 |
| 163 | PHE0006752_8521.pep | SRF-TF | 9 | 59 | 117.1 | 4.70E−32 |
| 163 | PHE0006752_8521.pep | K-box | 75 | 174 | 163.9 | 3.80E−46 |
| 160 | PHE0006774_8489.pep | CBFD_NFYB_HMF | 34 | 106 | 112 | 1.60E−30 |
| 161 | PHE0006778_8503.pep | CBFD_NFYB_HMF | 34 | 106 | 106.2 | 8.90E−29 |
| 164 | PHE0006779_8565.pep | CBFD_NFYB_HMF | 34 | 106 | 106.5 | 7.30E−29 |
| 162 | PHE0006780_8502.pep | CBFD_NFYB_HMF | 34 | 106 | 102.1 | 1.50E−27 |
| 165 | PHE0006781_8573.pep | CBFD_NFYB_HMF | 34 | 106 | 95.6 | 1.30E−25 |
| 174 | PHE0006858_8859.pep | SRF-TF | 9 | 59 | 115.3 | 1.60E−31 |
| 174 | PHE0006858_8859.pep | K-box | 74 | 173 | 148.4 | 1.70E−41 |
| 175 | PHE0006860_8863.pep | SRF-TF | 9 | 59 | 121.5 | 2.20E−33 |
| 175 | PHE0006860_8863.pep | K-box | 74 | 172 | 152.7 | 8.70E−43 |
| 177 | PHE0006951_9137.pep | zf-C2H2 | 152 | 175 | 20.1 | 0.0071 |
| 179 | PHE0006951_9173.pep | zf-C2H2 | 152 | 175 | 20.1 | 0.0071 |
| 176 | PHE0006955_9129.pep | SRF-TF | 9 | 59 | 95.2 | 1.80E−25 |
| 176 | PHE0006955_9129.pep | K-box | 85 | 175 | 22.5 | 5.10E−06 |
| 178 | PHE0006981_9158.pep | GRAS | 149 | 456 | 481 | 1.30E−141 |

TABLE 12
Description of Pfam domain

| | Accession | Gathering | |
|---|---|---|---|
| Pfam domain name | number | cutoff | Domain description |
| AP2 | PF00847.9 | 0 | AP2 domain |
| AT_hook | PF02178.8 | 3.6 | AT hook motif |
| AUX_IAA | PF02309.6 | −83 | AUX/IAA family |
| Auxin_resp | PF06507.3 | 25 | Auxin response factor |
| B3 | PF02362.12 | 26.5 | B3 DNA binding domain |
| CBFD_NFYB_HMF | PF00808.12 | 18.4 | Histone-like transcription factor (CBF/NF-Y) |
| | | | and archaeal histone |
| CCT | PF06203.4 | 25 | CCT motif |
| DUF296 | PF03479.4 | −11 | Domain of unknown function (DUF296) |
| EIN3 | PF04873.3 | −137.6 | Ethylene insensitive 3 |
| ELK | PF03789.3 | 25 | ELK domain |
| F-box | PF00646.21 | 13.6 | F-box domain |
| GATA | PF00320.16 | 28.5 | GATA zinc fincer |
| GRAS | PF03514.4 | −78 | GRAS family transcription factor |
| HLH | PF00010.15 | 8.2 | Helix-loop-helix DNA-binding domain |
| Homeobox | PF00046.18 | −4.1 | Homeobox domain |
| K-box | PF01486.7 | 0 | K-box region |
| KNOX1 | PF03790.3 | 25 | KNOX1 domain |
| KNOX2 | PF03791.3 | 25 | KNOX2 domain |
| Myb_DNA-binding | PF00249.19 | 2.8 | Myb-like DNA-hinding domain |
| NAM | PF02365.5 | −19 | No apical meristem (NAM) protein |
| PB1 | PF00564.13 | 12.3 | PB1 domain |
| RWP-RK | PF02042.5 | 25 | RWP-RK domain |
| Response_reg | PF00072.12 | 4 | Response regulator receiver domain |
| SBP | PF03110.5 | 25 | SBP domain |
| SRF-TF | PF00319.8 | 11 | SRF-type transcription factor (DNA-binding |
| | | | and dimerisation domain) |
| TCP | PF03634.3 | −38 | TCP family transcription factor |
| Tub | PF01167.7 | −98 | Tub family |
| bZIP_1 | PF00170.10 | 16.5 | bZIP transcription factor |
| bZIP_2 | PF07716.4 | 15 | Basic region leucine zipper |
| zf-B_box | PF00643.14 | 15.3 | B-box zinc finger |
| zf-C2H2 | PF00096.15 | 16.8 | Zinc finger, C2H2 type |
| zf-Dof | PF02701.5 | 25 | Dof domain, zinc finger |

### Example 10

### Selection of Transgenic Plants with Enhanced Agronomic Trait(s)

This example illustrates the preparation and identification by selection of transgenic seeds and plants derived from transgenic plant cells of this invention where the plants and seed are identified by screening for an enhanced agronomic trait imparted by expression of a protein selected from the group including the homologous proteins identified in Example 6. Transgenic plant cells of corn, soybean, cotton, canola, wheat and rice are transformed with recombinant DNA for expressing each of the homologs identified in Example 6. Plants are regenerated from the transformed plant cells and used to produce progeny plants and seed that are screened for enhanced water use efficiency, enhanced cold tolerance, increased yield, enhanced nitrogen use efficiency, enhanced seed protein and enhanced seed oil. Plants are identified exhibiting enhanced traits imparted by expression of the homologous proteins.

What is claimed is:

1. A plant cell nucleus with stably integrated, recombinant DNA construct, wherein said recombinant DNA constrict comprises a promoter that is functional in a plant cell and that is operably linked to a DNA segment encoding a protein comprising an amino acid sequence of SEQ ID NO: 177; and
wherein said recombinant DNA construct is stably integrated into a chromosome in said plant cell nucleus which is selected by screening a population or transgenic plants that have said recombinant DNA construct and an enhanced trait as compared to control plants that do not have said recombinant DNA construct in their nuclei; and
wherein said enhanced trait is selected from group of enhanced traits consisting of enhanced water use efficiency, enhanced cold tolerance, enhanced heat tolerance, enhanced high salinity tolerance, enhanced shade tolerance, increased yield, enhanced nitrogen use efficiency, enhanced seed protein and enhanced seed oil.

2. A recombinant DNA construct comprising a promoter that is functional in a plant cell and that is operably linked to a DNA segment that encodes:
a. at least one protein having an amino acid sequence comprising a Pfam domain module selected from the group consisting of Homeobox, Myb_DNA-binding::Myb_DNA-binding, Myb_DNA-binding, zf-Dof, zf-C2H2::zf-C2H2, AP2, Response_reg::Myb_DNA-binding, B3, B3::Auxin_resp::AUX_IAA, HLH, NAM, B3::B3, AUX_IAA, KNOX1::KNOX2::ELK, GRAS, AT_hook::AT_HOOK::DUF296, TCP, SBP; zf-C2H2, B3::Auxin_resp, EIN3, bZIP_{—}2, zf-B_box::zf-B_box, zf-B_box::CCT, RWP-RK::PB1, F-box::TUB, CBFD_NFYB_HMF, GATA, SRF-TF, K-box, and SRF-TF::K-box;
b. a protein comprising an amino acid sequence with at least 90% identity to a consensus amino acid sequence as set forth in SEQ ID NO: 2201;
c. a protein having an amino acid sequence having at least 70% identity to an amino acid sequence selected from the group consisting of SEQ ID NOs 96 through SEQ ID NO: 193;
or
d. a protein having an amino acid sequence selected from the group consisting of SEQ ID NO: 96 through SEQ ID NO: 193;
and wherein said recombinant DNA construct is stably integrated into a chromosome in a plant cell nucleus which is selected by screening a population of transgenic plants that have said recombinant DNA construct and an enhanced trait as compared to control plants that do not have said recombinant DNA construct in their nuclei; and wherein said enhanced trait is selected from group of enhanced traits consisting of enhanced water use efficiency, enhanced cold tolerance, enhanced heat tolerance, enhanced high salinity tolerance, enhanced shade tolerance, increased yield, enhanced nitrogen use efficiency, enhanced seed protein and enhanced seed oil.

3. A transgenic plant cell nucleus comprising a recombinant DNA construct of claim 2.

4. A transgenic plant cell having a plant cell nucleus of claim 3.

5. The transgenic plant cell of claim 4 wherein said transgenic plant cell is homozygous for said recombinant DNA construct.

6. The transgenic plant cell of claim 4 further comprising DNA expressing a protein that provides tolerance from exposure to an herbicide applied at levels that are lethal to a wild type of said plant cell.

7. The transgenic plant cell of claim 5 wherein said herbicide is a glyphosate, dicamba, or glufosinate compound.

8. A transgenic plant comprising a plurality of plant cells of claim 4.

9. The transgenic plant of claim 8 wherein said transgenic plant is homozygous for said recombinant DNA construct.

10. A transgenic seed comprising a recombinant DNA construct of claim 2.

11. The transgenic seed of claim 10 from a corn, soybean, cotton, canola, alfalfa, wheat or rice plant.

12. A transgenic pollen grain comprising a recombinant DNA construct of claim 2.

13. A method for manufacturing transgenic seeds that can be used to produce a crop of transgenic plants with an enhanced trait resulting from expression of a DNA segment in a plant cell nucleus comprising a recombinant DNA construct of claim 2, wherein said method comprises:
(a) providing a population of plants produced from a parental plant having a recombinant DNA construct of claim 2;
(b) screening, said population of plants for at least one of said enhanced trait and said recombinant DNA construct, wherein individual plants in said population can exhibit said trait at a level less than, essentially the same as or greater than the level that said trait is exhibited in control plants which do not contain said recombinant DNA construct, wherein said enhanced trait is selected from the group of enhanced traits consisting of enhanced water use efficiency, enhanced cold tolerance, enhanced heat tolerance, enhanced high salinity tolerance, enhanced shade tolerance, increased yield, enhanced nitrogen use efficiency, enhanced seed protein and enhanced seed oil;
(c) selecting from said population one or more plants that exhibit said trait at a level greater than the level that said trait is exhibited in control plants; and
(d) collecting seeds from selected plant selected from step c.

14. The method of claim 13, wherein said method further comprises:
(e) verifying that said recombinant DNA construct is stably integrated in said selected plants; and
(f) analyzing tissue of said selected plant to determine the expression of a protein having the function of a protein having an amino acid sequence selected from the group consisting of one of SEQ ID NO: 96 through SEQ ID NO: 193.

15. The method of claim 14 wherein said seed is corn, soybean, cotton, alfalfa, canola wheat or rice seed and said recombinant DNA construct is homozygous in said plant.

16. A method of producing hybrid corn seed comprising:
(a) acquiring hybrid corn seed from a herbicide tolerant corn plant which also has a stably-integrated, recombinant DNA construct of claim 2;
(b) producing corn plants from said hybrid corn seed, wherein a fraction of the plants produced from said hybrid corn seed is homozygous for said recombinant DNA construct, a fraction of the plants produced from said hybrid corn seed is hemizygous for said recombinant DNA construct, and a fraction of the plants produced from said hybrid corn seed has none of said recombinant DNA construct;
(c) selecting corn plants which are homozygous or hemizygous for said recombinant DNA construct by treating with an herbicide;
(d) collecting seeds from herbicide-treated-surviving corn plants and planting said seed to produce further progeny corn plants;
(e) repeating steps (c) and (d) at least once to produce an inbred corn line; and
(f) crossing said inbred corn line with a second corn line to produce hybrid corn seed.
